SEQUENCE LISTING (28 sequences)

SEQ ID NO: 1 - DNA, 20 bp, Artificial Sequence
  chemically synthesized PCR primer
gcgacccacacgtcaaacta

SEQ ID NO: 2 - DNA, 23 bp, Artificial Sequence
  chemically synthesized PCR primer
tcccttgatagacacaactcctc

SEQ ID NO: 3 - DNA, 21 bp, Artificial Sequence
  chemically synthesized PCR primer
actctgcgctggttgaaaaat

SEQ ID NO: 4 - DNA, 21 bp, Artificial Sequence
  chemically synthesized PCR primer
ggtggcatagcgaaccttgta

SEQ ID NO: 5 - DNA, 21 bp, Artificial Sequence
  chemically synthesized PCR primer
acttggggaccacctattcct

SEQ ID NO: 6 - DNA, 19 bp, Artificial Sequence
  chemically synthesized PCR primer
atcgccaatcagacgctcc

SEQ ID NO: 7 - DNA, 20 bp, Artificial Sequence
  chemically synthesized PCR primer
cggaacctgaggagagagtg

SEQ ID NO: 8 - DNA, 20 bp, Artificial Sequence
  chemically synthesized PCR primer
ctgtcagccaagctagggac

SEQ ID NO: 9 - DNA, 22 bp, Artificial Sequence
  chemically synthesized PCR primer
atggaagggaagtggttactgt

SEQ ID NO: 10 - DNA, 22 bp, Artificial Sequence
  chemically synthesized PCR primer
gctttgtaggtgacctttggag

SEQ ID NO: 11 - DNA, 20 bp, Artificial Sequence
  chemically synthesized PCR primer
acaccgaccaccgtatctca

SEQ ID NO: 12 - DNA, 23 bp, Artificial Sequence
  chemically synthesized PCR primer
ctcaggataatggtagccatgtc

SEQ ID NO: 13 - DNA, 18 bp, Artificial Sequence
  chemically synthesized PCR primer
cgcctccgatgtgttgga

SEQ ID NO: 14 - DNA, 22 bp, Artificial Sequence
  chemically synthesized PCR primer
cagtgcaatccacctttgctaa

SEQ ID NO: 15 - DNA, 22 bp, Artificial Sequence
  chemically synthesized PCR primer
tcgtcagagctgatgatgaagt

SEQ ID NO: 16 - DNA, 23 bp, Artificial Sequence
  chemically synthesized PCR primer
gcgtttaacccatccaactgaat

SEQ ID NO: 17 - DNA, 21 bp, Artificial Sequence
  chemically synthesized PCR primer
ttcctggtaaccgaatgctga

SEQ ID NO: 18 - DNA, 21 bp, Artificial Sequence
  chemically synthesized PCR primer
cctgaatctcggcgacttttt

SEQ ID NO: 19 - DNA, 19 bp, Artificial Sequence
  chemically synthesized PCR primer
ccacctctctatccgctgg

SEQ ID NO: 20 - DNA, 23 bp, Artificial Sequence
  chemically synthesized PCR primer
accaatgtgctaaccgtcttatt

SEQ ID NO: 21 - DNA, 23 bp, Artificial Sequence
  chemically synthesized PCR primer
tttggcaaatacaacccttcaga

SEQ ID NO: 22 - DNA, 21 bp, Artificial Sequence
  chemically synthesized PCR primer
gcagaagatactgtcaccacc

SEQ ID NO: 23 - DNA, 21 bp, Artificial Sequence
  chemically synthesized PCR primer
ctaccgagaacgagcctgatt

SEQ ID NO: 24 - DNA, 23 bp, Artificial Sequence
  chemically synthesized PCR primer
agccttccaattccttaaagcag

SEQ ID NO: 25 - DNA, 21 bp, Artificial Sequence
  chemically synthesized PCR primer
gaaccaggagttaagaacacg

SEQ ID NO: 26 - DNA, 20 bp, Artificial Sequence
  chemically synthesized PCR primer
aggcaacagtgtcagagtcc

SEQ ID NO: 27 - DNA, 21 bp, Artificial Sequence
  chemically synthesized PCR primer
gaaccaggagttaagaacacg

SEQ ID NO: 28 - DNA, 20 bp, Artificial Sequence
  chemically synthesized PCR primer
aggcaacagtgtcagagtcc

(19) United States
(12) Patent Application Publication
(10) Pub. No.: US 2024/0285728 A1
(43) Pub. Date: Aug. 29, 2024

(54) TARGETED PROTEIN DELIVERY TO MAMMALIAN EMBRYOS AND THERAPEUTIC APPLICATIONS USING EXOSOMES

(71) Applicants: Peixin YANG, Ellicott City, MD (US); ALBERT E. REECE, Lutherville, MD (US); University of Maryland, Baltimore, Baitimore, MD (US)

(72) Inventors: Peixin Yang, Ellicott City, MD (US); E. Albert REECE, Lutherville, MD (US)

(73) Assignee: University of Maryland, Baltimore, Baltimore, MD (US)

(21) Appl. No.: 18/568,036

(22) PCT Filed: Jun. 30, 2022
(86) PCT No.: PCT/US22/35715

§ 371 Date: Dec. 7, 2023

Related U.S. Application Data
(60) Provisional application No. 63/218,062, filed on Jul. 2, 2021.

(51) Int. Cl. A61K 38/17; A61K 9/00; A61K 9/51; A61K 38/18

(52) U.S. Cl. CPC A61K 38/1761; A61K 9/0019; A61K 9/5184; A61K 38/1825

(57) ABSTRACT

Methods for preventing neural tube defects in embryos of diabetic females via administration of Survivin-containing exosomes derived from Flk-1⁺ mesoderm progenitor cells are reported.

### STATEMENT OF FEDERALLY SPONSORED RESEARCH AND DEVELOPMENT

This invention was made with government support under Grant Numbers HL131737, HD100195, HD102206, DK083243, DK101972, and DK103024 awarded by the National Institutes of Health. The government has certain rights in the invention.

### BACKGROUND OF INVENTION

The US infant mortality rate, a basic measure of public health, is among the highest among developed countries, and birth defects are a major cause of infant mortality [1]. Neural tube defects (NTDs) are one of the leading types of birth defects and they are caused by many factors [2], one of them being pregestational maternal diabetes, a major non-genetic factor [3]. There are more than three million women in America, and 60 million worldwide, of reproductive age (18-44 years old) with diabetes, and these numbers are expected to double by 2030. However, the mechanism underlying pregestational maternal diabetes-induced birth defects is unknown.

One of the keys to preventing NTDs is understanding how normal embryonic development occurs, thereby revealing potential in-points of dysregulation that lead to birth defects and potential therapeutic targets. Diabetic embryopathy can be used as a specific example for understanding how altered normal developmental processes lead to birth defect formation [4]. Diabetic embryopathy is a devastating diabetic complication affecting both the mother and fetus. Hyperglycemia, the excess of glucose, is the major factor in mediating the teratogenicity of maternal diabetes, leading to NTD formation [5-8].

Preexisting maternal diabetes disrupts the process of vasculogenesis [9], which is initiated by the appearance of vascular endothelial growth factor receptor 2 (VEGFR2 or Flk-1, hereafter referred to as Flk-1)-positive hemangioblasts on mouse embryonic day 7 (E7) in the yolk sac mesoderm [10]. On E8 and later, maternal diabetes interrupts neurulation in the neuroectoderm, the innermost layer of the embryo, leading to neural tube defects (NTDs) [11-13]. Preliminary evidence suggests that there is crosstalk between components of early vasculopathy and those of late NTD formation, with the former being the proposed facilitators of the latter [14,15]. The spatial and temporal distinction between vasculogenesis and neurulation implies that maternal diabetes affects intercellular communication among the three germ layers, thereby leading to diabetic embryopathy.

Precise cell-to-cell communication among the three germ layers of the developing embryo is critical for normal embryogenesis. Soluble factors are conventional carriers for intercellular communication. However, exosomes, membrane-derived nanovesicles that mediate intercellular communication via functional cargos, may play important roles in embryonic development [16]. Functional exosome cargos include RNA and proteins [17]. The Survivin protein, an anti-stress, anti-apoptosis and pro-mitotic factor [18], is present in the membrane of exosomes derived from cancer cells [19]. Of note, the deletion of the Survivin gene, specifically in Flk-1⁺ progenitors during embryonic organogenesis, results in NTD formation [20], suggesting that Survivin produced by the Flk-1⁺ progenitors is packaged in exosomes and then acts on neuroepithelial cells in promoting neurulation. Neuroepithelial cells engulf these exosomes, and the Survivin in these exosomes abrogates maternal diabetes-induced cellular stress and excessive neuroepithelial cell apoptosis, major etiological factors for NTD induction in diabetic pregnancy [4,11,21-24]. Further experiments are required to clarify the role of Survivin in embryonic morphogenesis.

Survivin is implicated not only in development but also in the processes of carcinogenesis and aging [18]. How Survivin expression is regulated in Flk-1⁺ progenitors during development of an embryo may be very different than regulation in cancer and aging cells. It has been shown that growth factor-responsive mitogen-activated protein kinase signaling activates Survivin transcription [25], implicating growth factor regulation of Survivin expression during development. Indeed, maternal diabetes suppresses an array of vascular growth factors, including FGF2, leading to embryonic vasculopathy [9,15,26]. FGF2 derived from the endoderm acts on mesoderm Flk-1⁺ progenitors to promote embryonic vasculogenesis [27,28]. Therefore, it is thought that the augmenting effect of FGF2 on Flk-1⁺ progenitors transpires through the stimulation of Survivin transcription and subsequently enhances the production of Survivin-containing exosomes.

Maternal diabetes alters epigenetic modifications by increasing DNA methylation and suppressing histone acetylation during embryonic neurulation [29-31]. DNA hypermethylation accounts for the downregulation of neurulation essential genes in the neuroepithelium in diabetic pregnancy [29]. The mechanism underlying maternal diabetes-repressed vascular growth factor gene expression leading to embryonic vasculopathy is unknown. However, the green tea polyphenol epigallocatechin-3-gallate, which has a preventive effect on diabetic embryopathy through inhibition of DNA hypermethylation [29], rescues embryonic vasculopathy in diabetic pregnancy [32]. This evidence supports the hypothesis that DNA hypermethylation represses FGF2 in diabetic embryopathy.

Further research may reveal a role for Survivin in preventing NTDs in pregnant diabetic women, as well as means for safe and effective delivery of Survivin to suitable locations in the embryo. The present invention is directed to these and other important goals.

### BRIEF SUMMARY OF INVENTION

As reported herein, the present inventors have found that exosomal regulation and communication across the three germ layers mediates the essential role of vascular Flk-1⁺ progenitors in guiding neuroepithelial cells for proper neurulation during embryogenesis. Fibroblast growth factor (FGF2) in the endoderm, restrained by DNA methylation, activates Survivin expression in Flk-1⁺ progenitors, leading to the release of Survivin-containing exosomes. Neuroepithelial cells in the ectoderm internalize these Survivin-containing exosomes to ensure complete and proper neurulation.

The inventors further found that maternal diabetes suppresses FGF2 expression, leading to repression of Survivin and Survivin-containing exosomes, disrupting exosomal communication and resulting in NTD formation.

The present invention is based on these discoveries and generally encompasses therapeutic applications for mammalian embryos in mothers afflicted with diabetes.

More specifically, and in a first embodiment, the present invention is directed to methods based on the delivery of therapeutically-effective amounts of Survivin-containing exosomes to the embryos of pregnant diabetic women.

In a first aspect of this embodiment, the invention is directed to methods of preventing neural tube defects (NTDs) in an embryo of a pregnant diabetic female subject. These methods comprise administering to a pregnant diabetic female subject and/or an embryo of a pregnant diabetic female subject a therapeutically-effective amount of exosomes containing Survivin polypeptide.

In a second aspect of this embodiment, the invention is directed to methods of reducing diabetes-induced neuroepithelial cell apoptosis in an embryo of a pregnant diabetic female subject. These methods comprise administering to a pregnant diabetic female subject and/or an embryo of a pregnant diabetic female subject a therapeutically-effective amount of exosomes containing Survivin polypeptide.

In a third aspect of this embodiment, the invention is directed to methods of abrogating maternal diabetes-induced cellular stress in an embryo of a pregnant diabetic female subject. These methods comprise administering to a pregnant diabetic female subject and/or an embryo of a pregnant diabetic female subject a therapeutically-effective amount of exosomes containing Survivin polypeptide.

In a second embodiment, the present invention is directed to methods based on the delivery of therapeutically-effective amounts of FGF2 to the embryos of pregnant diabetic women.

In a first aspect of this embodiment, the invention is directed to methods of preventing NTDs in an embryo of a pregnant diabetic female subject. These methods comprise administering to a pregnant diabetic female subject and/or an embryo of a pregnant diabetic female subject a therapeutically-effective amount of fibroblast growth factor 2 (FGF2).

In a second aspect of this embodiment, the invention is directed to methods of reducing diabetes-induced neuroepithelial cell apoptosis in an embryo of a pregnant diabetic female subject. These methods comprise administering to a pregnant diabetic female subject and/or an embryo of a pregnant diabetic female subject a therapeutically-effective amount of FGF2.

In a third aspect of this embodiment, the invention is directed to methods of abrogating maternal diabetes-induced cellular stress in an embryo of a pregnant diabetic female subject. These methods comprise administering to a pregnant diabetic female subject and/or an embryo of a pregnant diabetic female subject a therapeutically-effective amount of FGF2.

In a third embodiment, the present invention is directed to methods based on the delivery of (i) therapeutically-effective amounts of Survivin-containing exosomes and (ii) therapeutically-effective amounts of FGF2 to the embryos of pregnant diabetic women.

In a first aspect of this embodiment, the invention is directed to methods of preventing neural tube defects (NTDs) in an embryo of a pregnant diabetic female subject. These methods comprise administering to a pregnant diabetic female subject and/or an embryo of a pregnant diabetic female subject (i) a therapeutically-effective amount of exosomes containing Survivin polypeptide and (ii) a therapeutically-effective amount of FGF2.

In a second aspect of this embodiment, the invention is directed to methods of reducing diabetes-induced neuroepithelial cell apoptosis in an embryo of a pregnant diabetic female subject. These methods comprise administering to a pregnant diabetic female subject and/or an embryo of a pregnant diabetic female subject (i) a therapeutically-effective amount of exosomes containing Survivin polypeptide and (ii) a therapeutically-effective amount of FGF2.

In a third aspect of this embodiment, the invention is directed to methods of abrogating maternal diabetes-induced cellular stress in an embryo of a pregnant diabetic female subject. These methods comprise administering to a pregnant diabetic female subject and/or an embryo of a pregnant diabetic female subject (i) a therapeutically-effective amount of exosomes containing Survivin polypeptide and (ii) a therapeutically-effective amount of FGF2.

In a fourth embodiment, the present invention is more generally directed to methods for delivering biological molecules to the embryos of pregnant diabetic women.

In a first aspect of this embodiment, the invention is directed to methods for delivering a biological molecule to neuroepithelial cells of an embryo of a pregnant female subject. These methods comprise administering to a pregnant female subject and/or an embryo of a pregnant female subject a therapeutically-effective amount of exosomes containing a biological molecule.

In relevant aspects and embodiments of the invention, the NTD may be, but is not limited to, failure of neural tube closure.

In relevant aspects and embodiments of the invention, the embryo has an age of from about E7 to E9.

In relevant aspects and embodiments of the invention, the exosomes are administered to the amniotic cavity of the embryo. Non-limiting examples of specific means of administration include microinjection.

In relevant aspects and embodiments of the invention, the exosomes are derived from Flk-1⁺ mesoderm progenitor cells.

In relevant aspects and embodiments of the invention, the therapeutically-effective amount of exosomes is a population of between 1×10⁶ and 1×10¹² exosomes. In certain aspects, the therapeutically-effective amount of exosomes is a population of 5×10⁹ exosomes.

In relevant aspects and embodiments of the invention, the female subject has gestational diabetes.

In relevant aspects and embodiments of the invention, the female subject is a female mammal.

In relevant aspects and embodiments of the invention, the therapeutically-effective amount of FGF2 is between 1 ng and 500 ng of FGF2.

In relevant aspects and embodiments of the invention, the biological molecule is one or more of a nucleotide, a oligonucleotide, polynucleotide, DNA, RNA, mRNA, microRNA, siRNA, cDNA, an amino acid, a peptide, a polypeptide, a protein, a lipid, a fatty acid, a glycolipid, a sterol, a monosaccharide, an oligosaccharide, a polysaccharide, a vitamin, a FDA approved drug, a growth factor, and anti-apoptotic factor, or a hormone.

The foregoing has outlined rather broadly the features and technical advantages of the present invention in order that the detailed description of the invention that follows may be better understood. Additional features and advantages of the invention will be described herein, which form the subject of the claims of the invention. It should be appreciated by those skilled in the art that any conception and specific embodiment disclosed herein may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that any description, figure, example, etc. is provided for the purpose of illustration and description only and is by no means intended to define the limits of the invention.

### BRIEF DESCRIPTION OF DRAWINGS

FIG. 1. Maternal diabetes suppresses FGF2 expression through DNA hypermethylation. (a and b) Abundance of FGF2 mRNA (a) and protein (b) in E7.5 conceptuses. (c) The CpG island (the light blue area) of the Fgf2 promoter. (d) The degree of methylation in the CpG island of Fgf2 promoter determined by Methylation-specific PCR (MSP) along with non-methylated specific PCR (NSP). (e) Methylated C (cytosine) frequencies in the 64 CpG sites of the CpG island after cloning CpG islands from the Fgf2 promoter for bisulfite sequencing. DNA isolated from E7.5 embryos of three dams (n=3) per group and DNA was cloned and sequenced twenty times per group. (f) Quantification of methylated cytosine from (e). (g) The methylation inhibitor RG108 restored Fgf2 promoter luciferase reporter activity suppressed by high glucose (25 mM glucose) in the yolk sac endoderm PYS2 cell line. Cells experiments were repeated three times (n=3). Experiments were performed using three embryos from three different dam per group (n=3). * indicates significant difference compared to other groups (P<0.05). ND: nondiabetic; DM: diabetes mellitus.

FIG. 2. FGF 2 overexpression ameliorates maternal diabetes-induced vasculopathy. (a) GFP was robustly expressed in the endoderm of E8.0 yolk sac. (b) mRNA abundance of Fgf2 and Fgfr. (c) Imaging and quantification of Flk-1⁺ progenitors in the yolk sac of E8.5 conceptuses, cell nuclei were counterstained with DAPI. (d) Imaging and quantification of blood island numbers in the yolk sac of E8.5 conceptuses (HE staining). (e) View of blood vessels in the yolk sac of E8.5 conceptuses. (f) CD31 staining in E8.5 embryos showing disruptive blood vessel formation in embryos exposed to maternal diabetes. (g) Blood vessel density determined by CD31 staining in the yolk sacs of E8.5 conceptuses. Experiments were performed using three embryos from three different dam per group (n=3). * indicates significant difference compared to other groups (P<0.05). ND: nondiabetic; DM: diabetes mellitus; WT: Wild-Type; FGF2: FGF2 transgenic mice.

FIG. 3. FGF2 overexpression alleviates maternal diabetes-induced apoptosis and NTD formation. (a) Protein abundance of FGF2 and (p)-FGFR in E8.5 embryos. (b) Protein abundance of cleaved caspase 3 and caspase 8 in E8.5 embryos. (c) Representative TUNEL assay imaging showing apoptotic cells (red dots) in the E8.5 neuroepithelium. Cell nuclei were stained with DAPI (blue), bars=30 μm. The bar graph shows quantification of TUNEL-positive cells. (d) Imaging of WT and FGF2 Tg overexpressing embryos. Experiments were performed using three embryos from three different dam per group (n=3). * indicates significant difference compared to other groups (P<0.05). ND: Nondiabetic; DM: Diabetes Mellitus; WT: Wild-Type; FGF2: FGF2 transgenic mice.

FIG. 4. FGF2 induces Survivin expression and enriches Survivin in exosomes. (a) Survivin mRNA and protein abundance in E8.5 Wild-Type (WT) and FGF2 Tg embryos from nondiabetic or diabetic dams. (b) Luciferase reporter activity driven by the Survivin promoter. Re-FGF2: human recombinant FGF2. (c) Survivin mRNA and protein expression in normal glucose (NG; 5 mM glucose) and high glucose (HG; 25 mM glucose) culture conditions with Re-FGF2. (d) Luciferase reporter activity driven by the Survivin promoter. si-FGF2: siRNA FGF2. (e) Survivin mRNA and protein expression. (f and g) Exosome number and Survivin protein expression in exosomes treated by Re-FGF2 (f) or si-FGF2 (g). (h and i) Exosome number and Survivin protein expression in exosomes of isolated E8 Flk-1⁺ progenitors treated by Re-FGF2 (h) or si-FGF2 (i). Cell culture experiments were performed three times (n=3). Cells experiments were repeated three times (n=3). Experiments were performed using three embryos from three different dam per group (n=3). * indicates significant difference (P<0.05) compared to other groups (P<0.05). ND: Nondiabetic; DM: Diabetes Mellitus; WT: Wild-Type; FGF2: FGF2 transgenic mice.

FIG. 5. Survivin overexpression ameliorates maternal diabetes-induced vasculopathy. (a) GFP was robustly expressed in Flk-1⁺ progenitors in the yolk sacs of E8.5 conceptuses. Survivin mRNA (b) and protein (c) expression in E8.5 embryos. (d) Imaging and quantification of Flk-1⁺ progenitors in the yolk sacs of E8.5 conceptuses. Cell nuclei were stained with DAPI (blue). (e) Imaging and quantification of blood island numbers in the yolk sacs of E8.5 conceptuses (HE staining). (f) Blood vessel density determined by CD31 staining in the yolk sacs of E8.5 conceptuses. (g) View of blood vessels in the yolk sacs of E8.5 conceptuses. (h) CD31 staining in E8.5 embryos showing disruptive blood vessel formation in embryos exposed to maternal diabetes. Experiments were performed using three embryos from three different dam per group (n=3). * indicates significant difference compared to other groups (P<0.05). ND: nondiabetic; DM: diabetes mellitus; WT: Wild-Type; Survivin: Survivin transgenic mice.

FIG. 6. Restoring Survivin expression in Flk-1⁺ progenitors reduces diabetes-induced apoptosis and NTD formation. (a) Protein levels of cleaved caspase 3 and caspase 8 in E8.5 embryos. (b) Representative TUNEL assay imaging showing apoptotic cells (red dots) in the E8.5 neuroepithelium and quantification of TUNEL positive cell numbers in the bar graph. Cell nuclei were stained with DAPI (blue), bars=30 μm. (c) Representative imaging of WT and Survivin transgenic overexpressing embryos showing an NTD embryo with an open neural tube. Experiments were performed using three embryos from three different dam per group (n=3). * indicates significant difference compared to other groups (P<0.05). ND: Nondiabetic; DM: Diabetes Mellitus; WT: Wild-Type. Survivin: Survivin transgenic overexpressing embryos.

FIG. 7. Survivin-enriched exosomes derived from Flk-1⁺ progenitors prevent diabetic embryopathy and the exosome inhibitor induces NTDs. (a) Exosomes (Exo) derived from Flk-1⁺ progenitors of E8.5 embryos. (b) Survivin protein expression in Flk-1⁺ progenitor exosomes from Survivin transgenic (Tg) and Wild-Type (WT) E8.5 embryos. (c) Labeled exosomes (green) from Flk-1⁺ progenitors but not C17.2 neural stem cells were up-taken by C17.2 neural stem cells. Cell nuclei were stained with DAPI (blue). (d) Flk-1⁺ progenitor exosomes (green) via in-utero amniotic microinjections were specifically up-taken by neuroepithelial cells of E8.0 embryos. (e to g) Survivin protein (e), cleaved caspase 8 and caspase 3 (f) and TUNEL-positive apoptotic cells (red). (g) After 36 hours exosome microinjections into E8.0 embryos. Bars=30 μm. (h) Numbers of normal and NTD embryos after exosome injections. Mock: mock vehicle (Veh) injections. n was indicated in Table 5. (i and j) Exosome profile (i), exosome numbers and CD63 expression (j) in E8.5 embryos after the exosome inhibitor GW4869 injections. (k) NTD rates in embryos. n was indicated in Table 6. (1) GW4869 concentrations in E8.5 embryos. (m). Morphology of embryos. (n) is a schematic diagram depicting the communication of Survivin-containing exosomes across germ layers, regulated by DNA hypermethylation of FGF2. Cells experiments were repeated three times (n=3). Experiments were performed using three embryos from three different dam per group (n=3). * indicates significant difference compared to other groups (P<0.05). ND: Nondiabetic; DM: Diabetes Mellitus; WT: Wild-Type; Veh: vehicle.

FIG. 8. FGF2 transgenic overexpression restores key vascular signaling adversely impacted by maternal diabetes. (a to d) Protein levels of (p)-AKT (a), BMP4 (b), VEGFR1 (c) and (p)-VEGFR2 (d) in E8.5 embryos. (e to g) mRNA levels of VEGFR1 (e), VEGFR2 (f) and BMP4 (g) in E8.5 embryos. Experiments were performed using three embryos from three different dams per group (n=3). * indicates significant difference compared to other groups (P<0.05). ND: nondiabetic; DM: diabetes mellitus; WT: Wild-Type; FGF2: FGF2 transgenic mice.

FIG. 9. FGF2 overexpression abrogates maternal diabetes-induced cellular stress. (a) Representative images of DHE staining. Red signals of DHE staining were observed in the V-shaped neuroepithelia of E8.5 embryos exposed to diabetes mellitus (DM). All cell nuclei were stained with DAPI (blue). Bars=30 μm. (b) Levels of lipid hydroperoxide (LPO) in E8.5 embryos. (c to f) Protein levels of (p)-PERK (c), (p)-eIF2α (d), (p)-IRE1α (e) and CHOP (f) in E8.5 embryos. (g) XBP1 mRNA splicing in E8.5 embryos. Arrows point to the actual size of the bands. (h) mRNA levels of Calnexin, GRP94, PDIA, BiP, IRE1α and CHOP. Experiments were performed using three embryos from three different dams per group (n=3). * indicates significant difference compared to other groups (P<0.05). ND: nondiabetic; DM: diabetes mellitus; WT: Wild-Type; FGF2: FGF2 transgenic mice.

FIG. 10. Survivin overexpression abrogates maternal diabetes-induced ER stress. (a to d) Protein levels of (p)-PERK (a), (p)-eIF2α (b), (p)-IRE1α (c) and CHOP (d) in E8.5 embryos. Experiments were performed using three embryos from three different dams per group (n=3). * indicates a significant difference (P<0.05) compared to the other groups. ND: nondiabetic; DM: diabetes mellitus; WT: Wild-Type; Survivin: Survivin transgenic mice.

FIG. 11. Neuroepithelial cells up-take Survivin-contained exosomes derived from Flk-1⁺ progenitors. (a) Representative imaging of Flk-1 or Nestin (the neural stem cell marker) or CD63 (the exosome marker) (red fluoresce) with myc staining (green) for myc-tagged Survivin in the yolk sacs (the top panel) and the neuroepithelia (the middle and the bottom panels) of E8.5 embryos. All cell nuclei were stained with DAPI (Blue). (b) Representative imaging of electron microscopy with CD63 (small-size silver grains) and myc (large-size silver grains) staining. Experiments were performed using three embryos from three different dams per group (n=3). * indicates significant difference compared to other groups (P<0.05). ND: nondiabetic; DM: diabetes mellitus; WT: Wild-Type; Survivin: Survivin transgenic mice.

FIG. 12. Exosomes from Flk-1⁺ cells but not from C17.2 neural stem cells abrogates maternal diabetes-induced ER stress. (a to d) Protein levels of (p)-PERK (a), (p)-IRE1α (b), (p)-eIF2α (c) and CHOP (d) in E8.5 embryos after intra-ammonic injections of exosomes in E8.0 embryos of diabetic dams. The group that did not receive any exosomes were mock injections of vehicle (PBS). Experiments were repeated three times (n=3). * indicates significant difference compared to other groups (P<0.05).

FIG. 13. GW4869 mimics maternal diabetes-induced ER stress and apoptosis. (a to d) Protein levels of (p)-PERK (a), (p)-IRE1α (b), (p)-eIF2α (c) and CHOP (d) in E8.5 embryos after GW4869 injections. (e) Protein levels of cleaved caspase 3 after GW4869 injections. (f). Protein levels of cleaved caspase 8 after GW4869 injections. Experiments were performed using three embryos from three different dams per group (n=3). * indicates significant difference compared to other groups (P<0.05). Veh: vehicle (saline) injections.

### DETAILED DESCRIPTION OF THE INVENTION

### I. Definitions

As used herein, “a” or “an” may mean one or more. As used herein when used in conjunction with the word “comprising,” the words “a” or “an” may mean one or more than one. As used herein “another” may mean at least a second or more. Furthermore, unless otherwise required by context, singular terms include pluralities and plural terms include the singular.

As used herein, “about” refers to a numeric value, including, for example, whole numbers, fractions, and percentages, whether or not explicitly indicated. The term “about” generally refers to a range of numerical values (e.g., +/−5-10% of the recited value) that one of ordinary skill in the art would consider equivalent to the recited value (e.g., having the same function or result). In some instances, the term “about” may include numerical values that are rounded to the nearest significant figure.

### II. The Present Invention

As summarized above, the present invention is generally directed to therapeutic techniques for mammalian embryos in mothers afflicted with diabetes.

As discussed in detail below, the present inventors have discovered a mechanism underlying maternal diabetes-induced neural tube defects (NTDs) in gestating embryos. They have found that during embryogenesis, proper communication among the three germ layers, facilitated by exosomes, is required for vasculogenesis and neurulation. In particular, fibroblast growth factor 2 (FGF2), produced by the endoderm, exerts effects on Flk-1⁺ progenitor cells in the mesoderm, leading to the release of exosomes enriched with the protein Survivin. These exosomes act on the neuroectoderm to prevent cellular stress and neuroepithelial cell apoptosis that can occur in diabetic women. This pathway can be inhibited under maternal diabetic conditions, resulting in vasculopathy, which, in turn, contributes to NTD formation.

The inventors have thus established that crosstalk occurs among components of the endoderm, mesoderm and neuroectoderm via FGF2 and Survivin-containing exosomes. This crosstalk is essential for successful neurulation. Maternal diabetes disrupts this crosstalk by suppressing FGF2 expression in the endoderm, and FGF2 reduction leads to the inhibition of Survivin expression in the Flk-1⁺ vascular progenitors of the mesoderm. In diabetic pregnancy, the exosomes derived from Flk-1⁺ progenitors, which contain significantly less Survivin than in a non-diabetic pregnancy, cannot propel neural tube closure, leading to NTD formation. These findings reveal an important role for the Survivin-containing exosomes produced by Flk-1⁺ progenitors in neurulation, and indicate that the endoderm factor FGF2 sustains Survivin expression, a process that is compromised by maternal diabetes.

The methods of the present invention, utilizing the discoveries of the inventors, comprise therapeutic techniques for mammalian embryos in mothers afflicted with diabetes.

As summarized above, in one embodiment the invention is directed to methods based on the delivery of therapeutically-effective amounts of Survivin-containing exosomes to the embryos of pregnant diabetic women.

In a first aspect of this embodiment, the invention is directed to methods of preventing neural tube defects (NTDs) in an embryo of a pregnant diabetic female subject. These methods comprise administering to a pregnant diabetic female subject and/or an embryo of a pregnant diabetic female subject a therapeutically-effective amount of exosomes containing Survivin polypeptide.

In a second aspect of this embodiment, the invention is directed to methods of reducing diabetes-induced neuroepithelial cell apoptosis in an embryo of a pregnant diabetic female subject. These methods comprise administering to a pregnant diabetic female subject and/or an embryo of a pregnant diabetic female subject a therapeutically-effective amount of exosomes containing Survivin polypeptide.

In a third aspect of this embodiment, the invention is directed to methods of abrogating maternal diabetes-induced cellular stress in an embryo of a pregnant diabetic female subject. These methods comprise administering to a pregnant diabetic female subject and/or an embryo of a pregnant diabetic female subject a therapeutically-effective amount of exosomes containing Survivin polypeptide.

In a second embodiment, the present invention is directed to methods based on the delivery of therapeutically-effective amounts of FGF2 to the embryos of pregnant diabetic women.

In a first aspect of this embodiment, the invention is directed to methods of preventing NTDs in an embryo of a pregnant diabetic female subject. These methods comprise administering to a pregnant diabetic female subject and/or an embryo of a pregnant diabetic female subject a therapeutically-effective amount of fibroblast growth factor 2 (FGF2).

In a second aspect of this embodiment, the invention is directed to methods of reducing diabetes-induced neuroepithelial cell apoptosis in an embryo of a pregnant diabetic female subject. These methods comprise administering to a pregnant diabetic female subject and/or an embryo of a pregnant diabetic female subject a therapeutically-effective amount of FGF2.

In a third aspect of this embodiment, the invention is directed to methods of abrogating maternal diabetes-induced cellular stress in an embryo of a pregnant diabetic female subject. These methods comprise administering to a pregnant diabetic female subject and/or an embryo of a pregnant diabetic female subject a therapeutically-effective amount of FGF2.

In a third embodiment, the present invention is directed to methods based on the delivery of (i) therapeutically-effective amounts of Survivin-containing exosomes and (ii) therapeutically-effective amounts of FGF2 to the embryos of pregnant diabetic women.

In a first aspect of this embodiment, the invention is directed to methods of preventing neural tube defects (NTDs) in an embryo of a pregnant diabetic female subject. These methods comprise administering to a pregnant diabetic female subject and/or an embryo of a pregnant diabetic female subject (i) a therapeutically-effective amount of exosomes containing Survivin polypeptide and (ii) a therapeutically-effective amount of FGF2.

In a second aspect of this embodiment, the invention is directed to methods of reducing diabetes-induced neuroepithelial cell apoptosis in an embryo of a pregnant diabetic female subject. These methods comprise administering to a pregnant diabetic female subject and/or an embryo of a pregnant diabetic female subject (i) a therapeutically-effective amount of exosomes containing Survivin polypeptide and (ii) a therapeutically-effective amount of FGF2.

In a third aspect of this embodiment, the invention is directed to methods of abrogating maternal diabetes-induced cellular stress in an embryo of a pregnant diabetic female subject. These methods comprise administering to a pregnant diabetic female subject and/or an embryo of a pregnant diabetic female subject (i) a therapeutically-effective amount of exosomes containing Survivin polypeptide and (ii) a therapeutically-effective amount of FGF2.

In a fourth embodiment, the present invention is more generally directed to methods for delivering biological molecules to the embryos of pregnant diabetic women.

In a first aspect of this embodiment, the invention is directed to methods for delivering a biological molecule to neuroepithelial cells of an embryo of a pregnant female subject. These methods comprise administering to a pregnant female subject and/or an embryo of a pregnant female subject a therapeutically-effective amount of exosomes containing a biological molecule.

In each of the relevant aspects and embodiments of the invention, the neural tube defect is one or more of failure of the neural tube to close, spina bifida, anencephaly, encephalocele, and iniencephaly.

In each of the relevant aspects and embodiments of the invention, the methods may be practiced on embryos undergoing neurulation.

In each of the relevant aspects and embodiments of the invention, the methods may be practiced on embryos ranging in age from E7 to E9. In certain aspects, the embryos are E8 in age when the methods of the invention are practiced.

In each of the relevant aspects and embodiments of the invention, the “preventing”, “reducing” and “abrogating” is at least 50%, in comparison to a subject not undergoing the one of the methods of the invention. Each of the “preventing”, “reducing” and “abrogating” may also be at least 50%, at least 60%, at least 70%, at least 80%, at least 90%, at least 95%, at least 99%, or 100%, in comparison to a subject not undergoing the one of the methods of the invention.

In each of the relevant aspects and embodiments of the invention, the female subject that is diabetic has pre-gestational diabetes, type 1 diabetes or type 2 diabetes. Exosomes are small, extracellular vesicles of 50-150 nm in diameter that mediate cell-to-cell communication. Because exosomes are noncytotoxic, have low immunogenicity, and are easy to store, and have a long lifespan and high cargo loading capacity, they have been proposed to be nanometric vehicles for therapeutic agent and gene delivery [42]. The mechanism underlying exosome-specific targeting is still unclear. One potential mechanism involves membrane receptors on the exosomes, which bind cell surface proteins of the recipient cells [43].

In each of the relevant aspects and embodiments of the invention, exosomes containing Survivin polypeptide are exosome isolated from Flk-1⁺ mesoderm progenitor cells and exosomes enriched with Survivin. In related aspects and embodiments of the invention where exosomes are used to deliver a biological molecule to neuroepithelial cells, the exosome may also be isolated from Flk-1⁺ mesoderm progenitor cells. Exosomes from Flk-1⁺ mesoderm progenitor cells can also be enriched with Survivin in a cell free system. The Flk-1⁺ progenitors may be isolated from embryos on E7.0 to E9.0. In certain aspects, the progenitors are isolated from embryos on E8.0. Once isolated, the exosomes may be harvested from the cells by means well known in the art, such as those described in the Examples herein.

The Survivin polypeptide, also called Baculoviral Inhibitor of apoptosis Repeat-Containing 5 or BIRC5, is a protein encoded by the BIRC5 gene in humans. Survivin is a member of the inhibitor of apoptosis (IAP) family proteins. It inhibits cell death and also promotes cell proliferation [18]. During embryonic development, Survivin is predominantly expressed in Flk-1⁺ progenitors and is essential for neurulation [20]. Survivin lacks intrinsic catalytic activity and thus functionally depends on its interactive partners [18]. Survivin blocks apoptosis through its interaction with other IAP proteins, inhibiting the caspase activation that initiates cell apoptosis [18]. Survivin is present on the surface of exosomes derived from cancer cells [19]. While exosomes derived from Flk-1⁺ progenitors contain abundant Survivin, the exact location of Survivin in these exosomes has not been determined. The stimulating effect of FGF2 on Survivin expression may rely on the activation of mitogen-activated protein kinase signaling, which has been shown to activate Survivin transcription [25]. The NCBI Reference Sequence for baculoviral IAP repeat-containing protein 5 isoform 1 [Homo sapiens] is NP_001159.2. The NCBI Reference Sequence for baculoviral IAP repeat-containing protein 5 isoform 2 [Homo sapiens] is NP_001012270.1. The NCBI Reference Sequence for baculoviral IAP repeat-containing protein 5 isoform 3 [Homo sapiens] is NP_001012271.1.

In each of the relevant aspects and embodiments of the invention, the “therapeutically-effective amount of exosomes containing Survivin polypeptide” is an amount of exosomes sufficient to achieve the goal of the method, i.e. preventing neural tube defects, reducing diabetes-induced neuroepithelial cell apoptosis, or abrogating maternal diabetes-induced cellular stress. The therapeutically-effective amount of exosomes, when the exosomes are isolated from Flk-1⁺ mesoderm progenitor cells, may also be defined as ranging from about 1×10⁶ to about 1×10¹² exosomes, from about 1×10⁷ to about 1×10¹¹ exosomes, or from about 1×10⁸ to about 1×10¹⁰ exosomes, or being about 1×10⁹ exosomes, 5×10⁹ exosomes, or 1×10¹⁰ exosomes.

Alternatively, the “therapeutically-effective amount of exosomes containing Survivin polypeptide” may be defined based on the total amount of Survivin polypeptide delivered to the embryo. For example, the therapeutically-effective amount of exosomes may contain about 1 ng to about 500 ng of Survivin polypeptide, or about 150 ng to about 500 ng of Survivin polypeptide.

The “therapeutically-effective amount of FGF2” is about 1 ng to about 500 ng of FGF2, or about 150 ng to about 500 ng of FGF2.

The female subjects on which the methods of the invention may be practiced are female mammals, e.g. a human, a non-human primate, horse, cow, goat, sheep, a companion animal, such as a dog, cat or rodent, or other mammal.

In each of the relevant aspects and embodiments of the invention, the biological molecule may be one or more of a nucleotide, a oligonucleotide, polynucleotide, DNA, RNA, mRNA, microRNA, siRNA, cDNA, an amino acid, a peptide, a polypeptide, a protein, a lipid, a fatty acid, a glycolipid, a sterol, a monosaccharide, an oligosaccharide, a polysaccharide, a vitamin, a FDA approved drug, a growth factor, and anti-apoptotic factor, or a hormone.

FGF2, also known as basic fibroblast growth factor (bFGF) and FGF-β, is a growth factor and signaling protein encoded by the FGF2 gene. The proteins is bound by and activates the fibroblast growth factor receptor (FGFR). The protein exhibits broad mitogenic and cell survival activities, and it has been implicated in a wide variety of biological processes, including embryonic development, cell growth, morphogenesis, tissue repair, tumor growth and invasion. The NCBI Reference Sequence for fibroblast growth factor 2 isoform 18 kDa [Homo sapiens] is NP_001348594.1. The NCBI Reference Sequence for fibroblast growth factor 2 isoform 34 kDa [Homo sapiens] is NP_001997.5.

In the methods of the present invention, the exosomes and/or FGF2 may be administered to the subject or the embryo at a frequency that includes a single administration to achieve the methods of the invention. Alternatively, a suitable number of administrations includes 1, 2, 3, 4, 5, 6, 7, 8, 9 or 10 administrations of the exosomes and/or FGF. When administered more than once, the frequency of administered may be every 15 minutes, every 30 minutes, every 45 minutes, every 60 minutes, every 75 minutes, every 90 minutes, every 105 minutes, or every 120 minutes, for example.

When administered to the subject, the exosomes and/or FGF2 may be formulated, for example, for oral, sublingual, intranasal, intraocular, rectal, transdermal, mucosal, pulmonary, topical or parenteral administration. The exosomes may be formulated in a suitable diluent, for example, phosphate buffered saline (PBS). Parenteral modes of administration include without limitation, intradermal, subcutaneous (s.c., s.q., sub-Q, Hypo), intramuscular (i.m.), intravenous (i.v.), intraperitoneal (i.p.), intra-arterial, intramedullary, intracardiac, intra-articular (joint), intrasynovial (joint fluid area), intracranial, intraspinal, and intrathecal (spinal fluids). Any known device useful for parenteral injection or infusion of drug formulations can be used to effect such administration.

The exosomes and/or FGF2 may be administered to the embryo via any means suitable in the art of delivery to an embryo. The exosomes may be formulated in a suitable diluent, for example, phosphate buffered saline (PBS). As a non-limiting example, intra-amniotic delivery via microinjections is a suitable means for administering exosome directly to an embryo and specifically targeting embryonic organs and cell types.

### III. Examples

### Materials and Methods

Animals and reagents. The procedures for animal use were approved by the University of Maryland School of Medicine Institutional Animal Care and Use Committee. Wild-type (WT) C57BL/6J mice were purchased from the Jackson Laboratory (Bar Harbor, ME). The DNA construct for the FGF2 transgenic mice (FGF2-Tg) was driven by the endoderm marker, transthyretin (TTR) from Dr. Terry A. Van Dyke (Department of Biological Sciences, University of Pittsburgh, PA). The DNA construct for the Survivin transgenic mice was driven by the Flk-1 promoter, which was cloned based on a previous report [44]. All Tg animal lines were generated in the Genome Modification Facility at Harvard University using the C57BL/6J background as previously described [11]. Streptozotocin (STZ; Sigma (St. Louis, MO)) was dissolved in sterile 0.1 M citrate buffer (pH 4.5).

Mouse models of diabetic embryopathy. The mouse model of diabetic embryopathy was described previously [4,22,36,45,46]. Briefly, ten-week old female mice were intravenously injected daily with 75 mg/kg STZ over two days to induce diabetes. Diabetes was defined as 12 hours fasting blood glucose level of ≥250 mg/dL. Male and female mice were paired at 3:00 PM, and day 0.5 (E0.5) of pregnancy was established by the presence of the vaginal plug at 8:00 AM the next morning. Female mice injected with vehicle served as the nondiabetic controls. On E7.5, E8.5, E9.5 or E10.5 mice were euthanized and conceptuses were dissected out of the uteri for biochemical and molecular analysis. The exosome inhibitor GW4869 (Sigma, St, Louis, MO) was injected daily to nondiabetic pregnant dams from E7.0 to E8.0. NTDs were examined at E10.5.

Cell culture and transfection. The C17.2 mouse neural stem cell line from the European Collection of Cell Culture (Salisbury, UK) and the yolk sac endoderm PYS2 cell line (ATCC® CRL2745™) were maintained in DMEM (5 mM glucose) supplemented with 10% FBS, 100 U/ml penicillin, and 100 mg/mL streptomycin at 37° C. in a humidified atmosphere of 5% CO₂. Mouse recombinant FGF2 (#3139-FB; R&D Systems, Minneapolis, MN) was used at a final concentration of 10 ng/ml. Lipofectamine 2000 (Invitrogen, Carlsbad, CA) was used according to the manufacturer's protocol for transfection of siFGF2 and control siRNA (Invitrogen, Carlsbad, CA) into the cells. After seeding for 12 h, cells were transfected with recombinant FGF2 and cultured in 1% FBS for 8 h, after which cells were cultured in 10% FBS and collected after 48 h for analysis.

Isolation of Flk-1+ endothelial progenitor cells. Flk-1⁺ progenitor cells were isolated from the E8 yolk sac. Yolk sacs were minced in phosphate-buffered saline PBS (Thermo Fisher Scientific, Waltham, MA), digested with 1 mg/ml of collagenase type I (Sigma, St Louis, MO) and incubated on an orbital shaker at 37° C. for 30-45 min. Following the collagenase treatment, cells were washed twice with growth medium (EBM-2, fetal bovine serum (10%), hFGF-basic (0.5 ml), hydrocortisone (0.2 ml), VEGF (0.5 ml), IGF-1 (0.5 ml), ascorbic acid (0.5 ml), hEGF (0.5 ml), GA-100 (0.5 ml)) before being plated. Cells were trypsinized (Lonza, Walkersville, MD) and sorted for Flk-1 with MACS microbeads (Miltenyi Biotec, Bergisch Gladbach, Germany) as per the kit instructions. The Flk-1⁺ progenitor cells were collected and cultured in growth medium as mentioned above.

Immunofluorescent staining. Tissue sections were fixed with 4% paraformaldehyde (pH7.4) for 30 min at room temperature, followed by permeabilization with 0.25% Triton-X100 (Sigma, St. Louis, MO) for 10 min. Samples were blocked for 30 min in PBS with 10% donkey serum and incubated with CD63 (Santa Cruz Biotechnology, Dallas, TX), Nestin (1:1000, Invitrogen, Carlsbad, CA), Flk-1 (1:200, Santa Cruz Biotechnology, Dallas, TX) and Myc-tag antibodies (1:200, Cell Signaling Technology, Danvers, MA) overnight at 4° C. After washing three times with PBS, samples were incubated with 488 and 594 secondary antibody (1:1000, Invitrogen, Carlsbad, CA) for 2 h, followed by DAPI (Invitrogen, Carlsbad, CA) cell nuclear counterstaining for 10 min, then mounted with aqueous mounting medium (Sigma, St Louis, MO). Confocal immunofluorescent images were recorded by a laser scanning microscope (LSM 510 META; Zeiss, Germany).

Exosome extraction and labelling. Cells and debris were removed from cell culture media via centrifugation at 2000×g for 30 minutes at 4° C. Supernatants containing the cell-free culture media were transferred to a new tube without disturbing the pellet. Total Exosome Isolation (#4478359, Invitrogen, Carlsbad, CA) at 0.5 volume of the collected supernatants was added to each tube. The cell culture media/reagent mixture was mixed well via vortex and pipetting up and down until it became a homogenous solution, and then was incubated at 4° C. overnight. After incubation, the samples were centrifuged at 10,000×g for 1 hour at 4° C. and the supernatants were discarded. Exosomes were contained in the pellet at the bottom of the tube (not visible in most cases). The pellet was re-suspended in 1 ml of 1×PBS, and NanoSight (#NS300, Malvern Panalytical) was used to measure the exosomes. The presence of exosomes was confirmed by Western blot using the exosomal marker CD63 (Santa Cruz Biotechnology, Dallas, TX). Exosomes were suspended in 1 ml Diluent C (Sigma, St Louis, MO) and labeled by incubating with PKH67 fluorescent dye (Sigma, St Louis, MO) for 5 min in the dark at room temperature. An equal volume of 1% BSA in PBS was added to each tube to stop the staining, with an additional incubation period of 1 minute to allow binding of excess dye. The labeled exosomes were separated from free dye using the Total Exosome Isolation kit (#4478359, Invitrogen, Carlsbad, CA).

In utero ultrasound-guided microinjection. E8.0 pregnant dams were anesthetized by 3% isoflurane (Vetone, Boise, ID) in 100% oxygen initially, followed by 2% isoflurane during the rest of the procedure. The dams were placed supine on a mouse handling table where the integrated warmer of the heating platform set to 37° C. and a rectal thermometer was put in place to monitor body temperature. The dams' eyes were covered with a lubricant to prevent drying of the sclera. Abdomen hairs of the dams were removed. A 2-cm ventral midline incision was made approximately 1 cm above the vagina to open the abdomen and peritoneum. The number of embryos was counted to ensure adequate record keeping of which embryos were injected. To stabilize the uterus, a modified Petri dish with a 3-cm hole cut into the middle covered by a thin silicone membrane with a small slit cut in the center was placed over the dams' abdomens. The uterine horns were gently pulled up through the silicon membrane of the dish, which contained 0.9% saline to prevent dehydration. Under ultrasound (Vevo 770, VisualSonics, Canada) guidance, the uterus was penetrated by a microinjection needle (#C060609, Coopersurgical) to reach the amniotic cavity. A total of 5×10⁹ exosomes suspended in 280 nl of PBS were injected into the amniotic cavity. Following one injection, the handling table was repositioned to image and inject the next embryo. After injecting the desired number of embryos, the uterine horns were placed back into the abdomen. The maternal abdomen was sutured closed.

Immunoblotting. Immunoblotting was performed as described by Yang at el [4]. Lysis buffer (Cell Signaling Technology, Danvers, MA) containing a protease inhibitor cocktail (Sigma, St Louis, MO) was used to extract protein. Equal amounts of protein and Precision Plus Protein Standards (Bio-Rad, Hercules, CA) were resolved by SDS-PAGE and transferred onto Immunobilon-P membranes (Millipore, Billerica, MA). Membranes were incubated in 5% nonfat milk for 45 minutes at room temperature, and then were incubated for 18 hours at 4° C. with the following primary antibodies at dilutions of 1:1000 in 5% nonfat milk. FGF2, phosphorylated (p-)FGFR, p-AKT, BMP4, mVEGFR1, p-VEGFR2, p-PERK, p-IRE1α, p-eIF2α, CHOP, caspase 8 and caspase 3. Following primary antibody incubation, membranes were washed with PBS and then exposed to HRP-conjugated related secondary antibodies at dilution of 1:10,000. Signals were detected using the SuperSignal West Femto Maximum Sensitivity Substrate kit (Thermo Fisher Scientific, Waltham, MA). To ensure that equivalent amounts of protein were loaded on the SDS-PAGE gel, membranes were stripped and incubated with a mouse antibody against R-actin (Abcam, Cambridge, MA). All experiments were repeated in triplicate with the use of independently prepared tissue lysates. The detailed antibody information is provided in Table 1.

TABLE 1

| Antibody | Antibody Source | Identifier |
|---|---|---|
| FGF2 | Millipore | 05-118 |
| p-FGFR | Invitrogen | 44-1140G |
| FGFR | Santa Cruz biotechnology | 9740s |
| p-AKT | Cell Signaling Technology | 4060s |
| AKT | Cell Signaling Technology | 9272s |
| BMP4 | Abcam | ab39973 |
| VEGFR1 | Invitrogen | 13687-1-AP |
| p-IRE1a | Invitrogen | PA1-16927 |
| IRE1a | Cell Signaling Technology | 3294 |
| p-PERK | Cell Signaling Technology | 3179 |
| PERK | Cell Signaling Technology | 3192 |
| p-eIF2a | Cell Signaling Technology | 3597 |
| eIF2a | Cell Signaling Technology | 2103 |
| CHOP | Cell Signaling Technology | 5554 |
| Caspase8 | Millipore | AB1879 |
| Caspase 3 | Millipore | AB1899 |
| CD31 | Abcam | ab28364 |
| FLK1 | Santa Cruz | sc6251 |
| nestin | Invitrogen | MA1-110 |
| survivin | Cell Signaling Technology | 2808s |
| myc | Cell Signaling Technology | 2272s |
| CD63 | Santa Cruz Biotechnology | sc-5275 |
| DAPI | Invitrogen | D1306 |
| Alexa Fluor 488-conjugated | Invitrogen | A-21202 |
| donkey anti-mouse IgG | | |
| secondary antibody | | |
| Alexa Fluor 488-conjugated | Invitrogen | A-21206 |
| donkey anti-rabbit IgG | | |
| secondary antibody | | |
| Alexa Fluor 594-conjugated | Invitrogen | A-21203 |
| donkey anti-mouse IgG | | |
| secondary antibody | | |
| Alexa Fluor 594-conjugated | Invitrogen | A-21207 |
| donkey anti-rabbit IgG | | |
| secondary antibody | | |
| β-actin | Abcam | ab8224 |

Real-time PCR (RT-PCR). mRNA was isolated from embryos using the RNeasy Mini kit (Qiagen, Hilden, Germany), and then reversely transcribed using the high-capacity cDNA archive kit (Applied Biosystem, Grand Island, NY). Real time-PCR for Fgf2, Fgfr, Bmp4, Vegfr2, mVegfr1, Calnexin, GRP94, PDIA, BiP, IRE1α, CHOP, Survivin and β-actin were performed using the Maxima SYBR Green/ROX qPCR Master Mix assay (Thermo Fisher Scientific, Waltham, MA) in the Step One Plus system (Applied Biosystem, Grand Island, NY). All primer sequences were listed in Table 2.

TABLE 2

| | | Primer | SEQ |
|---|---|---|---|
| Primer | | Sequences | ID |
| Name | Direction | (5'-3') | NO: |
| Fgf2 | Forward primer | GCGACCCACA | 1 |
| | | CGTCAAACTA | |
| | Reverse primer | TCCCTTGATA | 2 |
| | | GACACAACTC | |
| | | CTC | |
| Fgfr | Forward primer | ACTCTGCGCT | 3 |
| | | GGTTGAAAAA | |
| | | T | |
| | Reverse primer | GGTGGCATAG | 4 |
| | | CGAACCTTGT | |
| | | A | |
| BiP | Forward primer | ACTTGGGGAC | 5 |
| | | CACCTATTCC | |
| | | T | |
| | Reverse primer | ATCGCCAATC | 6 |
| | | AGACGCTCC | |
| CHOP | Forward primer | CGGAACCTGA | 7 |
| | | GGAGAGAGTG | |
| | Reverse primer | CTGTCAGCCA | 8 |
| | | AGCTAGGGAC | |
| Calnexin | Forward primer | ATGGAAGGGA | 9 |
| | | AGTGGTTACT | |
| | | GT | |
| | Reverse primer | GCTTTGTAGG | 10 |
| | | TGACCTTTGG | |
| | | AG | |
| IRE1α | Forward primer | ACACCGACCA | 11 |
| | | CCGTATCTCA | |
| | Reverse primer | CTCAGGATAA | 12 |
| | | TGGTAGCCAT | |
| | | GTC | |
| PDIA | Forward primer | CGCCTCCGAT | 13 |
| | | GTGTTGGA | |
| | Reverse primer | CAGTGCAATC | 14 |
| | | CACCTTTGCT | |
| | | AA | |
| GRP94 | Forward primer | TCGTCAGAGC | 15 |
| | | TGATGATGAA | |
| | | GT | |
| | Reverse primer | GCGTTTAACC | 16 |
| | | CATCCAACTG | |
| | | AAT | |
| BMP4 | Forward primer | TTCCTGGTAA | 17 |
| | | CCGAATGCTG | |
| | | A | |
| | Reverse primer | CCTGAATCTC | 18 |
| | | GGCGACTTTT | |
| | | T | |
| VEGFR1 | Forward primer | CCACCTCTCT | 19 |
| | | ATCCGCTGG | |
| | Reverse primer | ACCAATGTGC | 20 |
| | | TAACCGTCTT | |
| | | ATT | |
| VEGFR2 | Forward primer | TTTGGCAAAT | 21 |
| | | ACAACCCTTC | |
| | | AGA | |
| | Reverse primer | GCAGAAGATA | 22 |
| | | CTGTCACCAC | |
| | | C | |
| survivin | Forward primer | CTACCGAGAA | 23 |
| | | CGAGCCTGAT | |
| | | T | |
| | Reverse primer | AGCCTTCCAA | 24 |
| | | TTCCTTAAAG | |
| | | CAG | |
| β-Actin | Forward primer | GAACCAGGAG | 25 |
| | | TTAAGAACAC | |
| | | G | |
| | Reverse primer | AGGCAACAGT | 26 |
| | | GTCAGAGTCC | |

Tissue section staining. Nondiabetic wild-type (WT), nondiabetic Tg, diabetic WT and diabetic Tg embryos were collected for a morphological examination at E10.5 and fixed in methacarn (methanol, 60%; chloroform, 30%; and glacial acetic acid, 10%). Embryos were dehydrated in alcohol and embedded in paraffin, then cut into 5-μm sections. After deparaffinization and rehydration, they were stained with hematoxylin and eosin (H&E), and imaged under a Nikon Ni-U microscope (Nikon, Tokyo, Japan).

Blood island quantification. 5 μm cross (vertical) sections of E8.5 conceptuses were stained by H&E. Yolk sac blood islands were counted and expressed against the yolk sac circumference. The yolk sac in an E8.5 conceptus cross section is near-circular, so it was assumed to be a circle for calculating circumference. Ten sections of each conceptus were used and the data were averaged. Five conceptuses from different pregnant dams in each group were used to determine the numbers of blood islands in yolk sacs.

Blood vessel density measurement. E9.5 conceptuses were fixed with 4% paraformaldehyde in PBS overnight at 4° C. For immunostaining analyses, controls were processed by omitting the primary antibody. The rat anti-mouse PECAM-1 antibody at a dilution of 1:200 (Abcam, Cambridge, MA) was used to stain the whole conceptuses. Samples were incubated in ABC solution (Elite ABC kit, Vector Laboratories) for 30 min and then with the stable diaminobenzidine substrate solution (Vector Laboratories, Burlingame, CA). Yolk sacs were removed from the conceptuses and mounted on positively charged slides. Embryos were examined directly. PECAM-1 positive structures (vessel area) in the yolk sac and embryo were determined by capturing images and analyzing the PECAM-1-stained areas with the NIH ImageJ software (Version 1.62, National Institutes of Health, Bethesda, MD) by setting a consistent threshold for all slides. The PECAM-1-positive area was expressed as pixels-squared per high-power field.

Dihydroethidium (DHE) staining. DHE staining was used for immunofluorescent detection of superoxide. DHE reacts with superoxide that binds to cellular components such as protein and DNA, and manifested with bright red fluorescence. E8.5 embryos were fixed in 4% Paraformaldehyde (PFA) (Thermo Fisher Scientific, Waltham, MA) for 30 minutes, washed for 5 min each for three times with PBS, and then embedded in optimal cutting temperature compound (OCT) (Sakura Finetek, Torrance, CA). 10 μm frozen embryonic sections were incubated with 1.5 μM DHE for 5 min at room temperature and then washed for 3 times with PBS. Sections were counterstained with DAPI and mounted with aqueous mounting medium (Sigma, St Louis, MO).

Lipid hydroperoxide (LPO) quantification. The degree of lipid peroxidation, an index of oxidative stress, was quantitatively assessed by the LPO assay by using the Calbiochem Lipid Hydroperoxide Assay Kit (Milliproe, Bedford, MA) following the manufacture's manual. Briefly, E8.5 embryos were homogenized in HPLC-grade water (Thermo Fisher Scientific, Waltham, MA). The lipid hydroperoxides of each embryo tissue were extracted by deoxygenated chloroform, and then measured by the absorbance of 500 nm after reaction with chromogen. The results were expressed as μM lipid hydroperoxides per microgram protein. Protein concentrations were assessed by the BioRad DC protein assay kit (BioRad, Hercules, CA).

Detection of XBP1 mRNA splicing. RNA was extracted from E8.5 embryos and reverse-transcribed to cDNA using the QuantiTect Reverse Transcription Kit (Qiagen, Hilden, Germany). The PCR primers for XBP1 were as follows: forward, 5′-GAACCAGGAGTTAAGAACACG-3′ (SEQ ID NO:27) and reverse, 5′-AGGCAACAGTGTCAGAGTCC-3′ (SEQ ID NO:28). If no XBP1 mRNA splicing occurred, a 205 bp band was produced. When XBP1 splicing occurred, a 205 bp band and a 179 bp main band were produced.

TUNEL assay. TUNEL assay was performed using the ApopTag Fluorescein in Situ Apoptosis Detection kit (#S7165, Millipore, Billerica, MA) as previously described [4]. Briefly, 5-μm frozen embryonic sections were fixed with 4% PFA in PBS and incubated with TUNEL reaction agents. Three embryos from three different dams (n=3) in each group were used, and three sections per embryo were examined. TUNEL-positive cells in an area (about 200 cells) of the neuroepithelium were counted. The percentage of TUNEL-positive cells was calculated as a fraction of the total cell number, multiplied by 100 and averaged within the sections of each embryo.

Statistical analyses. Data were presented as means±standard errors (SE). In animal studies, experiments were repeated at least three times, and embryonic samples from each replicate were from different dams. Statistical differences were determined by one-way analysis of variance (ANOVA) using the SigmaPlot 12.5 (SigmaStat, San Jose, CA). In one-way ANOVA analysis, Tukey test was used to estimate the significance of the results (P<0.05). The Chi square test and Fisher Exact test were used to estimate the significance of NTD incidence.

### Results

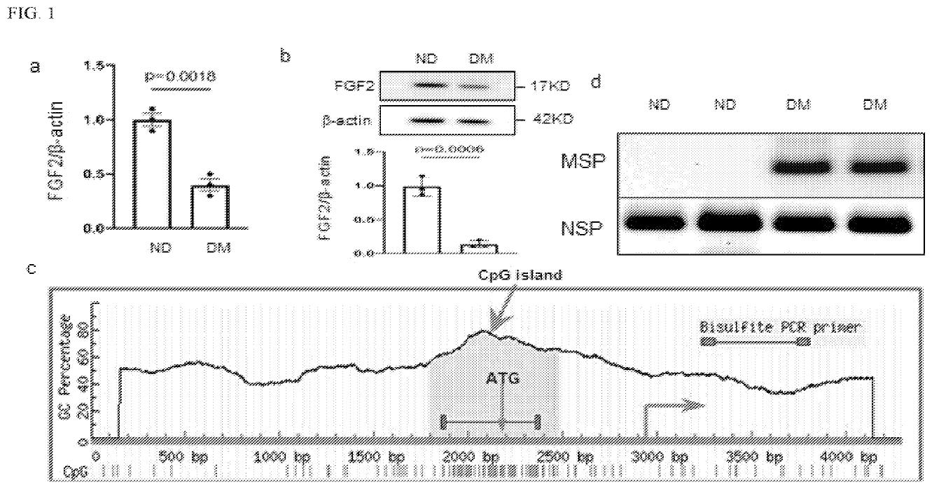

Maternal diabetes-induced DNA methylation inhibits FGF2 expression. It was hypothesized that molecular defects in the vascular progenitors of early vasculogenesis trigger the neuroepithelial cell dysfunction leading to NTD formation. To test this hypothesis, the expression of fibroblast growth factor 2 (FGF2), which induces mesoderm epiblast cells to differentiate into Flk-1⁺ progenitors in the mesoderm [33,34], was examined. On E7.5, maternal diabetes transiently reduced the abundance of FGF2 mRNA and protein (FIGS. 1, A and B). DNA hypermethylation typically occurs at CpG islands in the promoter regions of target genes to silence gene expression. A CpG island in the Fgf2 gene promoter was identified (FIG. 1 C), and methylation-specific PCR showed increased methylation in this CpG island (FIG. 1 D). The methylated cytosine frequency of the 64 CpG sites in the CpG island of Fgf2 was increased more than four-fold by maternal diabetes (FIGS. 1, E and F). Treatment with the DNA methylation inhibitor RG108 [35] restored Fgf2 promoter-driven luciferase activity suppressed by high glucose (FIG. 1 G). These findings collectively support the hypothesis that the high glucose in maternal diabetes represses FGF2 expression in the endoderm through DNA hypermethylation.

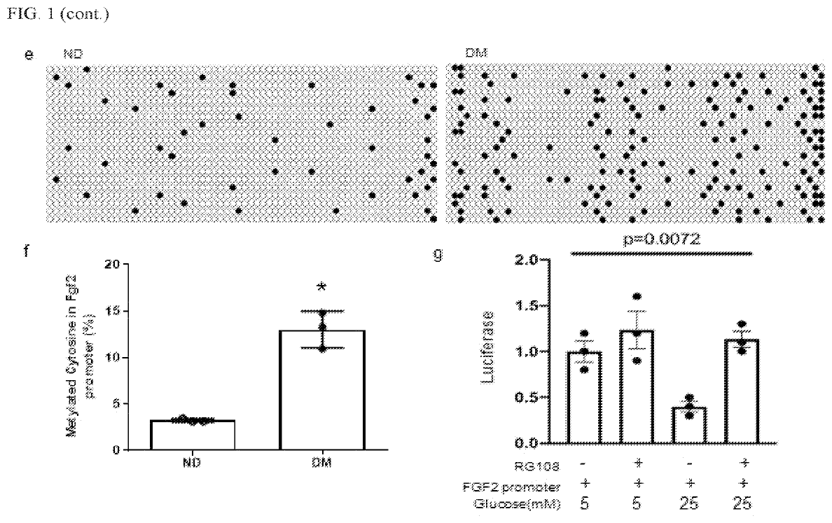

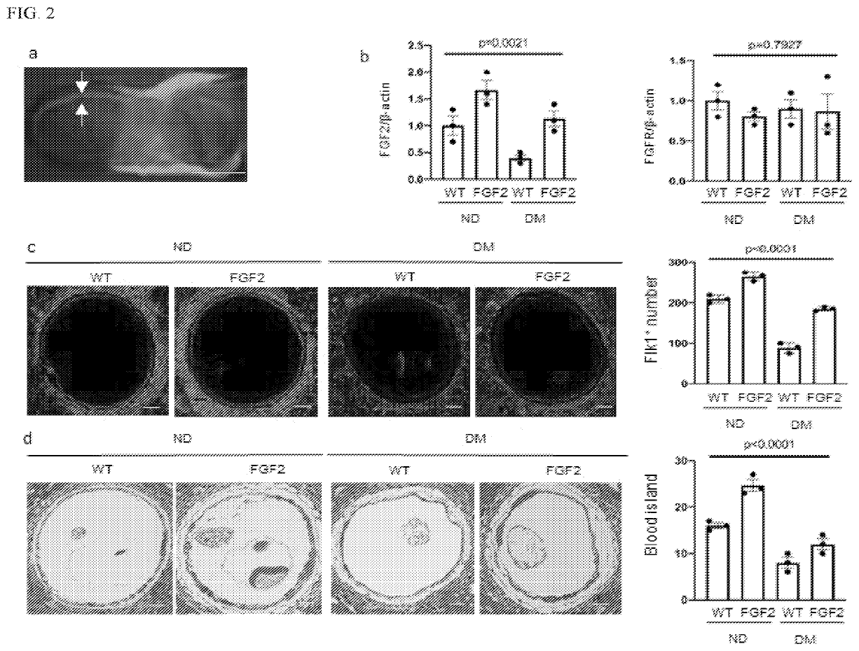

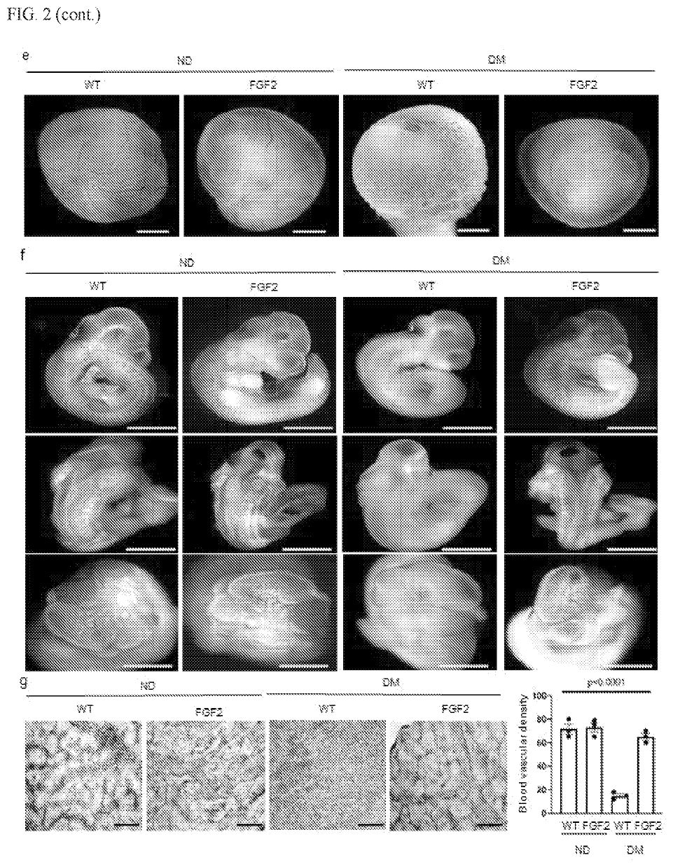

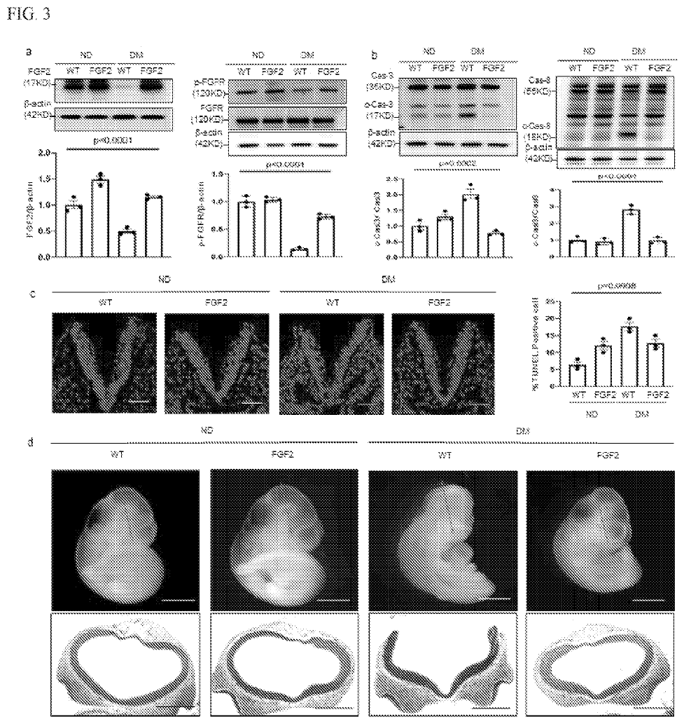

FGF2 restoration ameliorates embryonic vasculopathy in diabetic pregnancy. To determine whether FGF2 reduction is responsible for vasculopathy in diabetic pregnancy, a transgenic (Tg) mouse line was generated in which the Fgf2 open reading frame and a separate nuclear GFP were driven by the promoter of the embryonic endoderm marker transthyretin (TTR). TTR-driven transgene expression was evident in the endoderm on E5.75 and later. GFP was evident in the endoderm of embryos on E8 (FIG. 2 A). FGF2-expressing Tg embryos had significantly higher levels of FGF2 than the wild-type (WT) embryos under nondiabetic conditions, which effectively led to a reversal of maternal diabetes-suppressed FGF2 expression and FGF receptor phosphorylation (FIG. 3 A). In addition, FGF2 overexpression restored key vascular signaling pathways impaired by maternal diabetes (FIG. 8, A to G). The reduced abundance of key vascular signaling intermediates p-AKT, p-VEGFR2 and BMP4 by maternal diabetes was recovered by FGF2 overexpression and reached the level of the nondiabetic controls (FIGS. 8, A, B, D, F and G). The increased abundance of VEGFR1, a negative regulator of embryonic vasculogenesis, was blunted in the FGF2-expressing Tg embryos (FIGS. 8, C and E). Maternal diabetes impacted the whole process of embryonic vasculogenesis. The total number of Flk-1⁺ progenitors was reduced by maternal diabetes (FIG. 2 C). A key process during embryonic vasculogenesis is the formation of blood islands by Flk-1⁺ progenitors. Consistent with the reduced number of Flk-1⁺ progenitors, blood island formation on E8.5 was significantly decreased in conceptuses exposed to diabetes (FIG. 2 D). The reversal of FGF2 reduction restored the number of Flk-1⁺ progenitors and blood islands (FIGS. 2, B and C) and rescued vascular defects in the yolk sac and the embryo (FIGS. 2, E and F), thus preventing the reduction of blood vessel density in the yolk sac (FIG. 2 G).

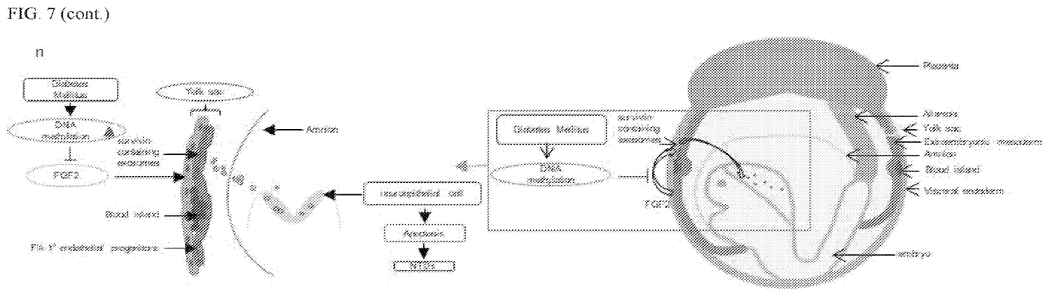

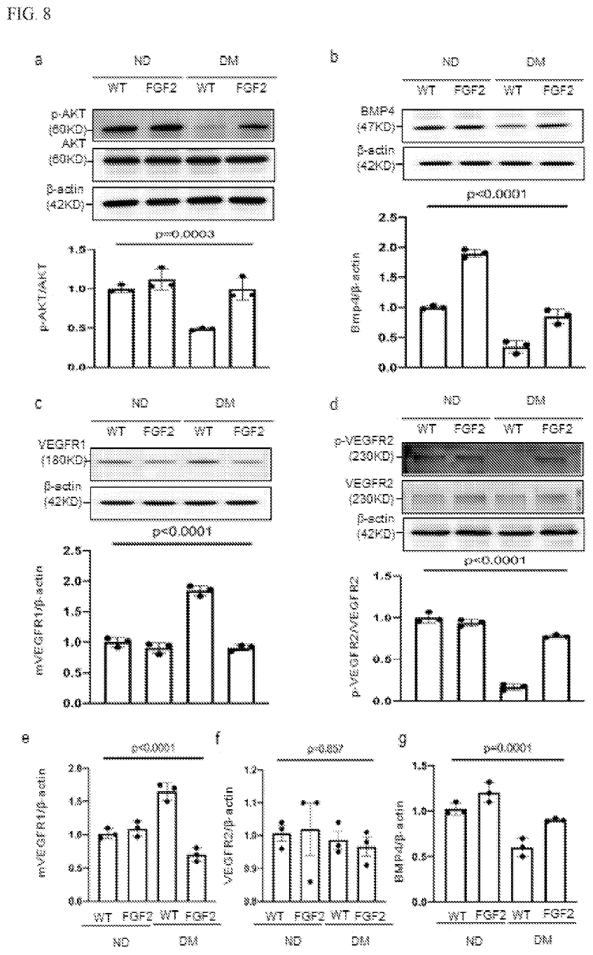

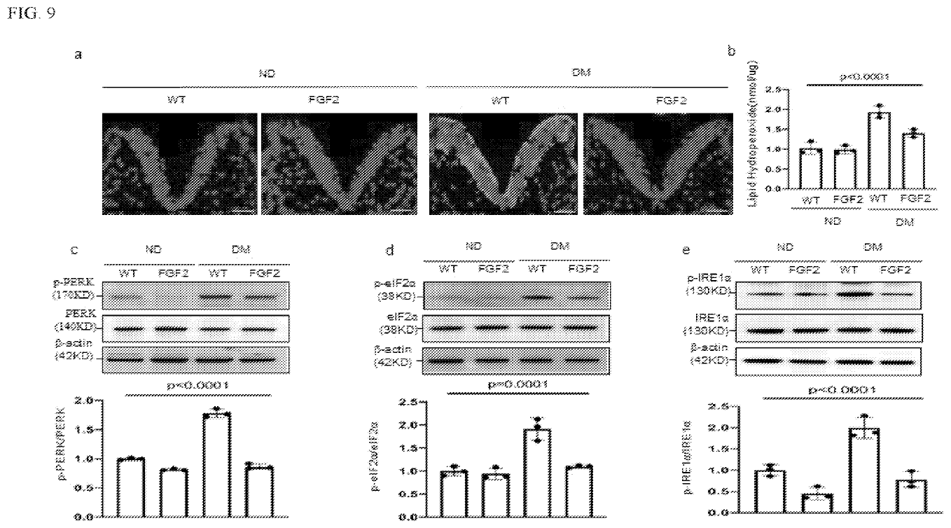

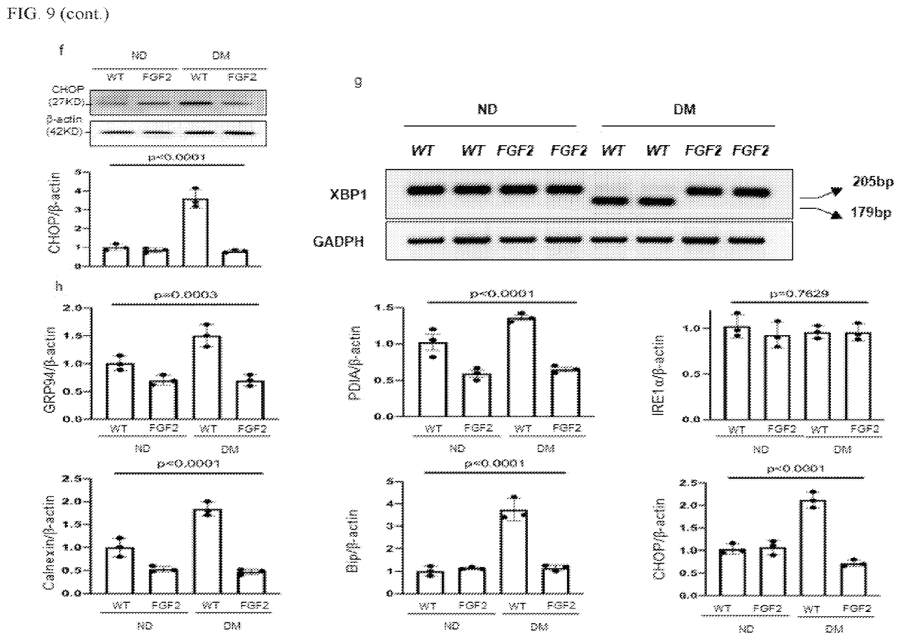

FGF2 restoration inhibits ER stress and NTD formation. Next, whether correcting vascular defects via FGF2 would mitigate the diabetes-induced stress pathway in the neuroepithelium was examined. Oxidative stress-induced endoplasmic reticulum (ER) stress that leads to apoptosis is manifested during failed neurulation during diabetic pregnancy [21,36,37]. Superoxide was abundant in the neuroepithelia of the WT embryos in diabetic dams but was diminished in the neuroepithelia of the WT embryos in nondiabetic dams and FGF2-expressing Tg embryos in nondiabetic and diabetic dams (FIG. 9 A). Maternal diabetes-induced increase in lipid peroxidation was also diminished in the FGF2-expressing Tg embryos (FIG. 9 B). Elevated abundance of the ER stress protein markers p-PERK, p-eIF2α, p-IRE1α and C/EBP homologous protein (CHOP) by maternal diabetes was abrogated in FGF2-expressing Tg embryos (FIG. 9, C to F). X-box binding protein 1 (XBP1) mRNA splicing, another indicator of ER stress, was induced by maternal diabetes and diminished in FGF2-expressing Tg embryos (FIG. 9 G). Furthermore, increased ER chaperone gene expression is also indicative of ER stress. The abundance of Calnexin, GRP94, PDIA, BiP and CHOP mRNA was significantly increased by maternal diabetes compared to levels in the WT embryos from nondiabetic dams and was abrogated by FGF2 overexpression (FIG. 9 H). Cleavage of the initiator caspase, caspase 8, and the executor caspase, caspase 3, was repressed by FGF2 overexpression under maternal diabetic conditions (FIG. 3 B). Correspondingly, the number of apoptotic cells in the neuroepithelia of the WT embryos from diabetic dams was higher than that in the WT embryos from nondiabetic dams, and FGF2 overexpression inhibited maternal diabetes-induced apoptosis (FIG. 3 C). These findings support the hypothesis that restoring FGF2 expression in the endoderm blocks maternal diabetes-induced cellular stress and apoptosis in the neuroepithelium.

To assess whether restoring FGF2 during vasculogenesis ameliorates maternal diabetes-induced NTDs, NTD formation on E10.5 was examined. FGF2 overexpression did not affect embryonic development as indicated by the WT and FGF2-overexpressing E10.5 embryos being morphologically indistinguishable (FIG. 3 D, Table 3). Further analysis indicated a significantly lower NTD incidence in the FGF2-expressing Tg embryos than in the WT counterparts that developed under diabetic conditions (FIG. 3 D, Table 3). It was observed that 23.1% of the WT embryos in diabetic dams exhibited NTDs, which was significantly higher than that of the WT embryos in nondiabetic dams (0%) (FIG. 3 D, Table 3), whereas only 5.7% of the FGF2-expressing Tg embryos (5.7%) exhibited NTDs (FIG. 3 D, Table 3).

TABLE 3

| | Glucose | | | NTD |
|---|---|---|---|---|
| | level | Geno- | Em- | Embryos |
| Experimental group | (mg/dl) | type | bryos | (NTD rate) |
| ND-FGF2-Tg male x | 136.7 ± 30.3 | WT | 56 | 0 (0.0%) |
| WT female (17 litters) | | FGF2-Tg | 58 | 1 (1.7%) |
| DM-FGF2-Tg male x | 388.4 ± 21.4 | WT | 52 | 12 (23.1%) * |
| WT female (17 litters) | | FGF2-Tg | 53 | 3 (5.7%) |

NTD, neural tube defect;
ND: nondiabetic;
DM: diabetes mellitus;
WT: Wild-Type;
Tg: transgenic.
* indicates significant difference compared to other groups (P < 0.05) analyzed by the Fisher's Exact test.

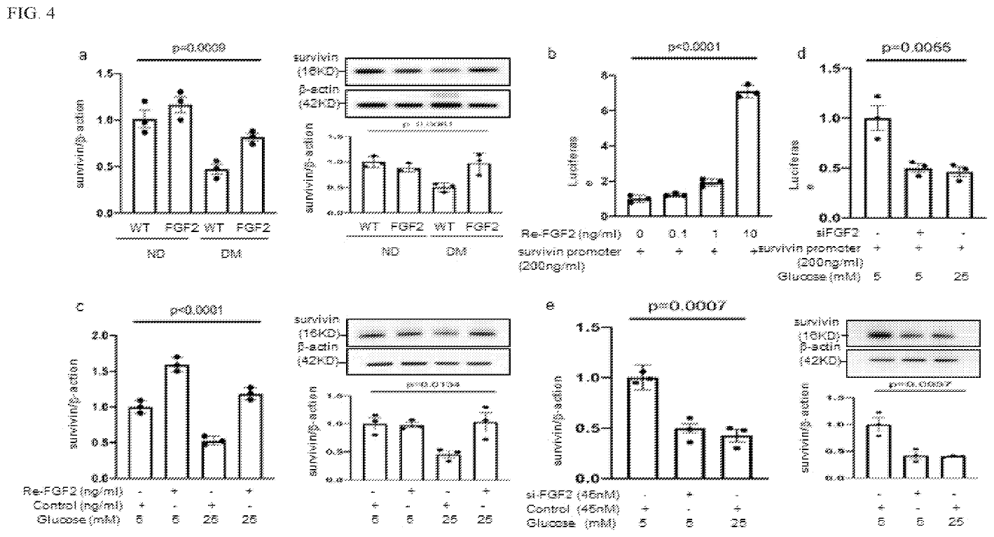

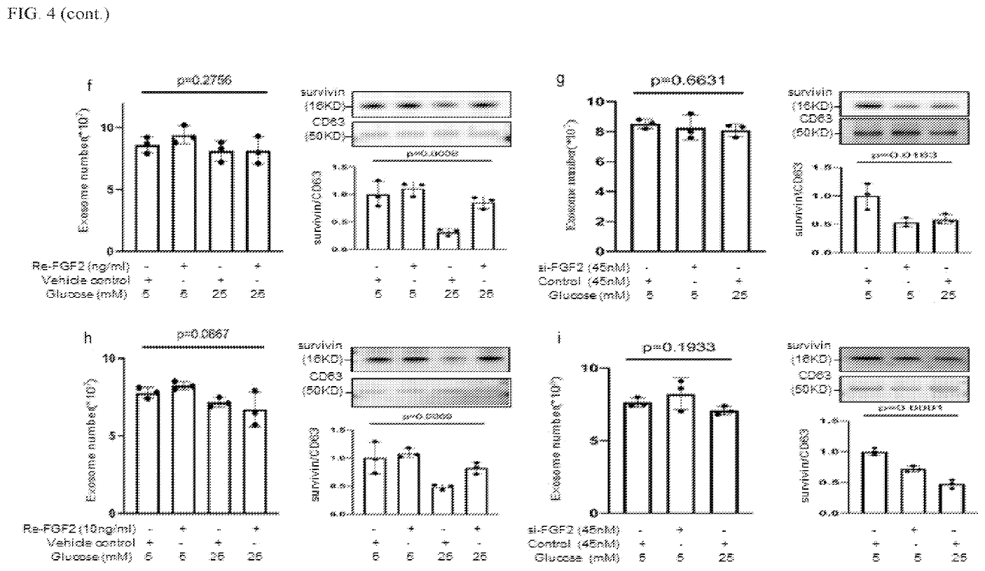

Survivin is the FGF2 target gene in Flk-1⁺ progenitors. Next, the target gene of FGF2 in the mesoderm was identified. Deleting the gene Survivin, which encodes the inhibitor of apoptosis protein [38] in vascular endothelial progenitor cells, results in NTDs [20]. Survivin also combats cellular stress [39]. For these reasons, it was proposed that Survivin is the FGF2 responsive gene in Flk-1⁺ progenitors in the mesoderm. Restoring FGF2 expression reversed maternal diabetes-induced suppression of Survivin mRNA and protein expression in the developing embryo (FIG. 4 A). FGF2 stimulated luciferase reporter activity driven by the Survivin promoter (FIG. 4 B) and normalized Survivin mRNA and protein downregulation induced by high glucose (FIG. 4 C). FGF2 knockdown mimicked high glucose in repressing Survivin promoter activity (FIG. 4 D) and in suppressing Survivin mRNA and protein expression (FIG. 4 E).

FGF2 abrogates the reduction in Survivin in the cargo of exosomes from Flk-1+ progenitors. Exosomes, small extracellular vesicles (50-150 nm), mediate cell-to-cell communication [40]. Survivin has been demonstrated to be incorporated as exosomal functional cargo [19]. High glucose did not affect the number of exosomes produced by a yolk sac cell line but significantly decreased the abundance of Survivin in the exosomal cargo (FIG. 4 F). FGF2 overexpression rescued Survivin expression in exosomal cargo that had been suppressed by high glucose (FIG. 4 F). In contrast, FGF2 knockdown mimicked high glucose in that it reduced Survivin levels in the exosomal cargo but did not affect the cell production of exosomes (FIG. 4 G). To determine the direct effect of high glucose on embryonic Flk-1⁺ progenitors, Flk-1⁺ progenitors were isolated on E8. High glucose suppressed Survivin expression in the exosomes produced by isolated E8 Flk-1⁺ progenitors, and FGF2 abolished this suppression (FIG. 4 H). Survivin protein abundance was significantly decreased in the exosomes derived from Flk-1⁺ progenitors of embryos in diabetic dams compared with the level in the embryos in nondiabetic dams, and this decrease was prevented by FGF2 transgene expression (FIG. 4 I). Maternal diabetes did not affect the exosome production ability of Flk-1⁺ progenitors (FIG. 41). These findings collectively suggest that FGF2 is essential for Survivin expression and its inclusion in the exosomal cargo of Flk-1⁺ progenitors.

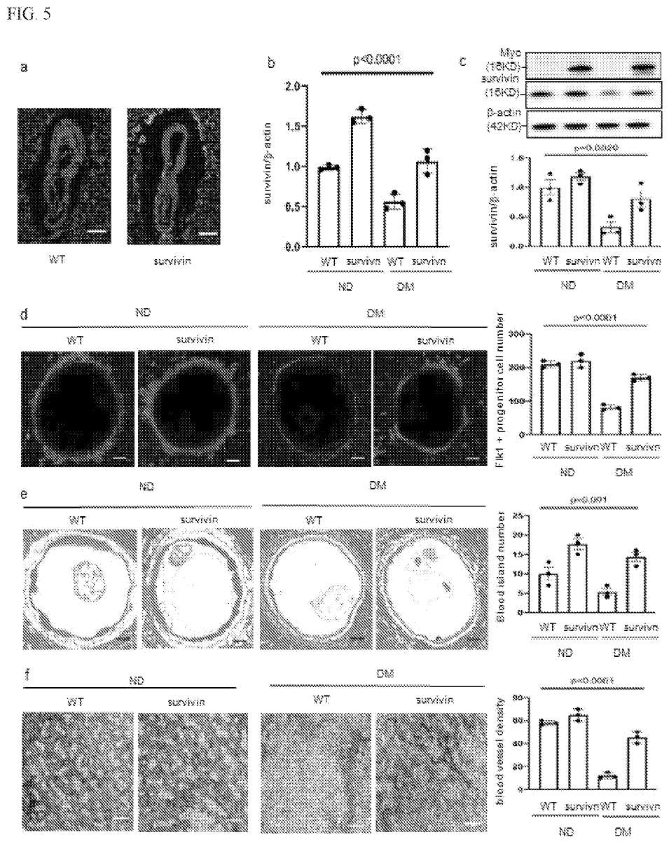

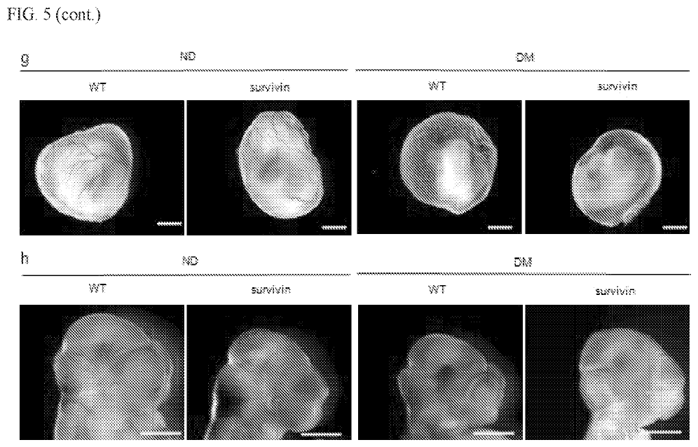

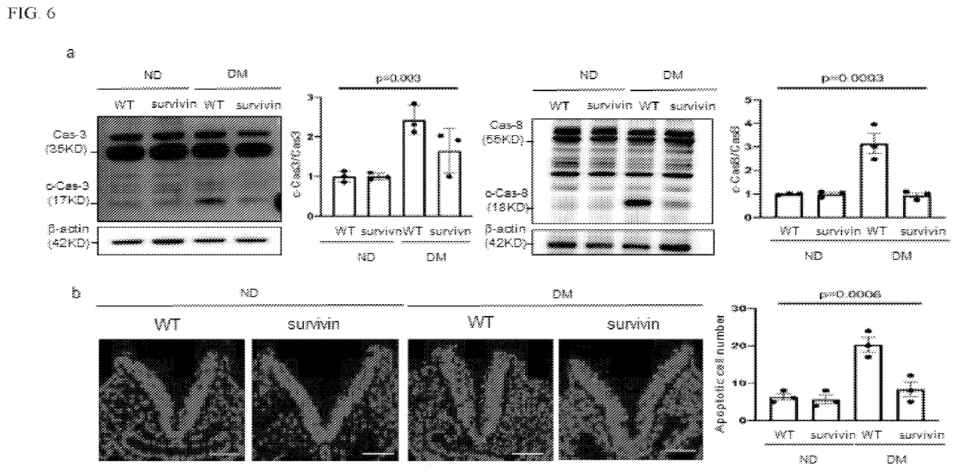

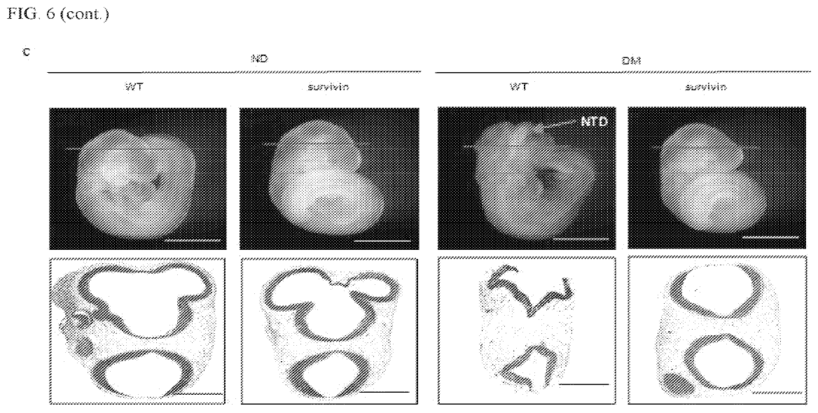

Survivin restoration in Flk-1+ progenitors ameliorates vasculopathy and prevents NTD formation. To determine the role of Survivin reduction in diabetic embryopathy, a transgenic mouse line was created in which GFP (myc-tagged Survivin) was specifically expressed in Flk-1⁺ progenitors (FIG. 5 A). Survivin transgenic overexpression restored Survivin expression that had been suppressed by maternal diabetes during vasculogenesis (FIGS. 5 B and C). Survivin overexpression in the Flk-1⁺ progenitors ameliorated maternal diabetes-induced NTDs on E10.5 (FIG. 6 C, Table 4).

TABLE 4

| | Glucose | | | NTD |
|---|---|---|---|---|
| | level | Geno- | Em- | Embryos |
| Experimental group | (mg/dl) | type | bryos | (NTD rate) |
| ND Survivin-Tg | 123.9 ± 28.5 | WT | 47 | 0 (0.0%) |
| male x WT female | | Survivin- | 42 | 0 (0.0%) |
| (11 litters) | | Tg | | |
| DM Survivin-Tg | 426.8 ± 26.7 | WT | 63 | 17 (26.98%) * |
| male x WT female | | Survivin- | 61 | 7 (11.48%) |
| (17 litters) | | Tg | | |

NTD, neural tube defect;
ND: nondiabetic;
DM: diabetes mellitus;
WT: Wild-Type;
Tg: transgenic.
* indicates significant difference compared to other groups (P < 0.05) analyzed by the Fisher's Exact test.

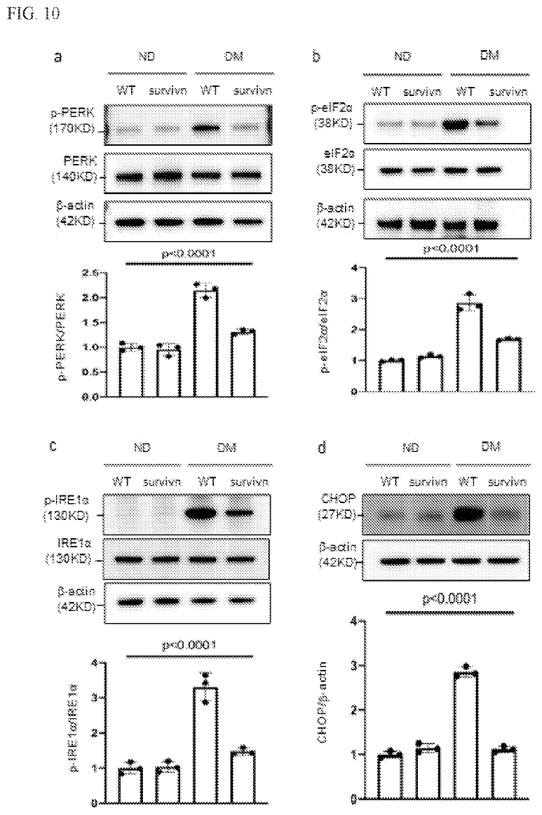

Because ER stress is an important factor in the pathogenesis of NTDs in diabetic pregnancy [11], ER stress markers were evaluated to address the effect of Survivin transgenic overexpression in Flk-1⁺ progenitors on cellular stress in neuroepithelial cells. The increase in ER stress markers, phosphorylated PERK, eIF2α and IRE1α, and total CHOP, induced by maternal diabetes, was diminished by Survivin transgenic overexpression in neurulation stage embryos (FIG. 10, A to D). ER stress was shown to lead to caspase 8-dependent apoptosis in the neuroepithelium [36]. The cleavage of caspase 3 and 8 was also abrogated by Survivin transgenic overexpression (FIG. 6 A). Thus, under diabetic conditions, the number of apoptotic cells in the developing neuroepithelium was reduced to a number similar to that in the nondiabetic group (FIG. 6 B).

Next, the cell autonomous effect of Survivin was investigated by examining Flk-1⁺ progenitor-initiated vasculogenesis. The total number of Flk-1⁺ progenitors was reduced by maternal diabetes, and restoring Survivin expression in Flk-1⁺ progenitors sustained their quantity under diabetic conditions (FIG. 5 D), supporting a cell autonomous effect of Survivin in Flk-1⁺ progenitors. Blood island number and vessel density in Survivin-overexpressing conceptuses in diabetic dams reached a quantity similar to those in conceptuses in nondiabetic dams (FIG. 5 E to H). Therefore, Survivin in Flk-1⁺ progenitors exerts a dual functional role in embryonic development: a cell autonomous effect in vasculogenesis and a paracrine effect on neurulation through the exosomal communication of Flk-1⁺ progenitors in the mesoderm and neuroepithelial cells in the neuroectoderm.

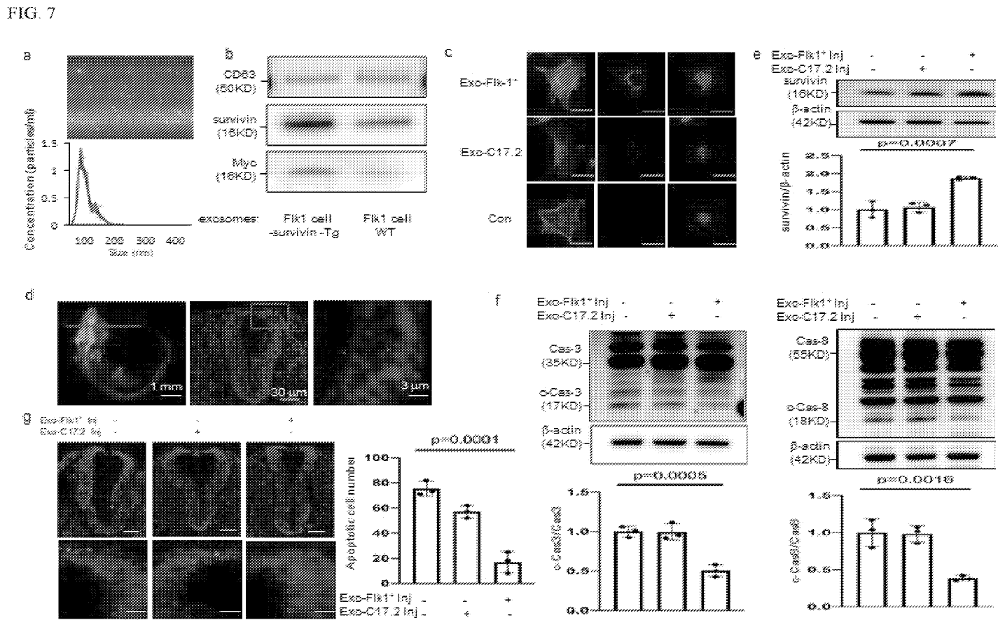

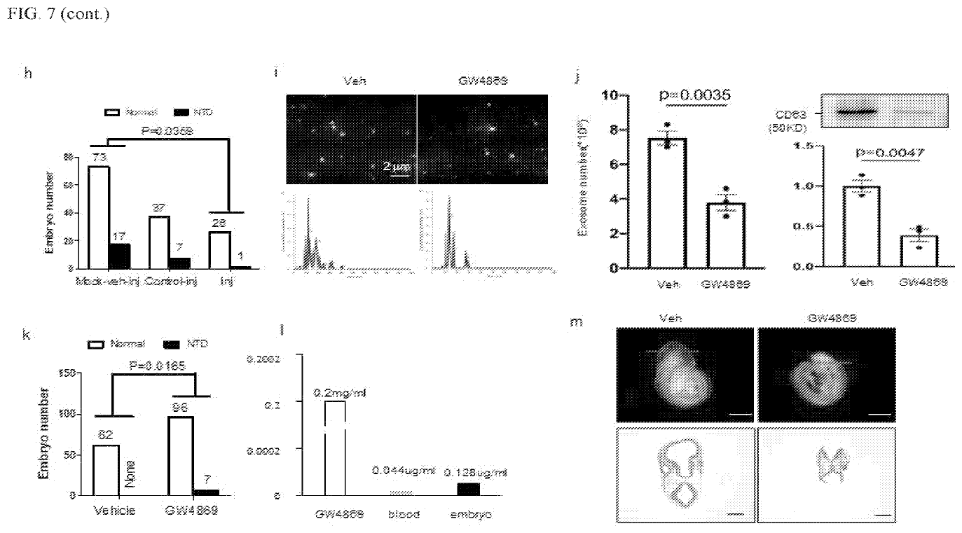

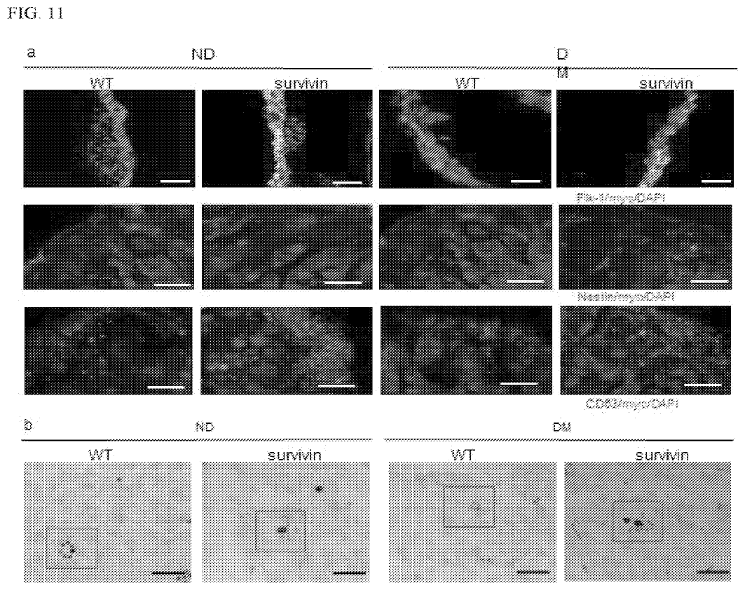

Survivin-containing exosomes derived from Flk-1+ progenitors target neuroepithelial cells. To further elucidate the role of exosomal communication involving Survivin in neurulation, Flk-1⁺ progenitors were isolated from embryos on E8.0 and harvested exosomes produced by these cells (FIG. 7 A). The exosomes derived from Flk-1⁺ progenitors in Survivin-expressing Tg embryos had enriched levels of Survivin (FIG. 7 B). The Flk-1⁺ progenitor exosomes could be effectively internalized by cultured neural stem cells (FIG. 7 C). The myc-tagged Survivin in the Tg conceptuses was found in the Flk-1⁺ progenitors in the E8.0 yolk sac and in the neuroepithelial cells (or neural stem cells) along with the exosome marker CD63 (FIG. 11 A). Electron microscopy silver staining showed the presence of the exosome marker CD63 in the engulfed myc-tagged Survivin (FIG. 11 B), supporting the hypothesis that neuroepithelial cells take up Survivin-containing exosomes derived from Flk-1⁺ progenitors.

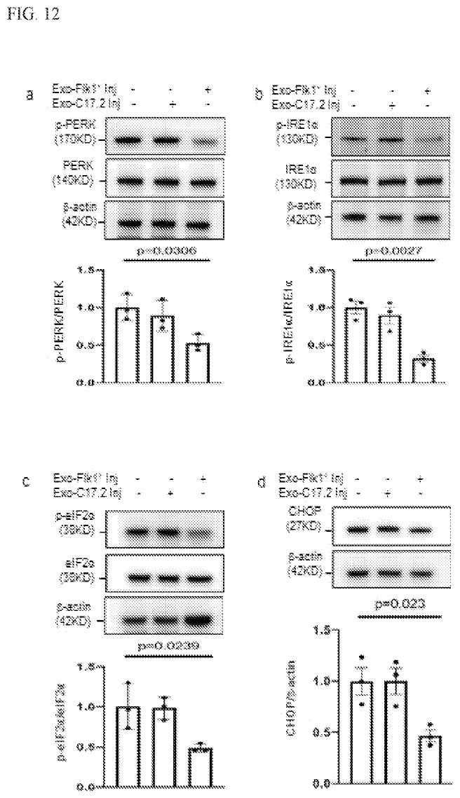

Intra-amniotic delivery of Survivin-containing exosomes inhibits NTD formation. The functional significance of Survivin-containing exosomes derived from E8.0 Flk-1⁺ progenitors was also demonstrated by the intra-amniotic delivery of these exosomes to neurulation stage embryos on E8.0. The Survivin-containing exosomes were specifically internalized by the neuroepithelium (FIG. 7 D), restored embryonic Survivin expression (FIG. 7 e), inhibited ER stress (FIG. 12), and blocked caspase 3 and 8 cleavage and neuroepithelial cell apoptosis under diabetic conditions (FIGS. 7 F and G). However, exosomes from neural stem cells did not efficiently target neural stem cells (FIG. 7 C), did not restore Survivin expression (FIG. 7 E), could not block ER stress (FIG. 12), and did not inhibit caspase activation and apoptosis (FIG. 7 f and g). Thus, Survivin-containing exosomes derived from Flk-1⁺ progenitors significantly reduced maternal diabetes-induced NTDs (FIG. 7 H, Table 5). In contrast, delivery of exosomes derived from neural stem cells without Survivin enrichment had no inhibitory effect on NTD formation in diabetic pregnancy (FIG. 7 H).

TABLE 5

| | Glucose | | | NTD |
|---|---|---|---|---|
| Experimental | level | | | Embryos |
| group | (mg/dl) | Genotype | Embryos | (NTD rate) |
| DM-WT male x | 416.15 ± 53.1 | Non-injection | 73 | 17 (23.29%) |
| WT female | | injection | 26 | 1 (3.85%) * |
| (20 litters) | | Con-injection | 37 | 7 (18.92%) |

NTD, neural tube defect;
DM: diabetes mellitus;
WT: Wild-Type.
* Indicates significant difference (P < 0.05) when compared to other groups analyzed by the Fisher's Exact test. 7 embryos absorbed after injection, 3 embryos absorbed with non-injection.

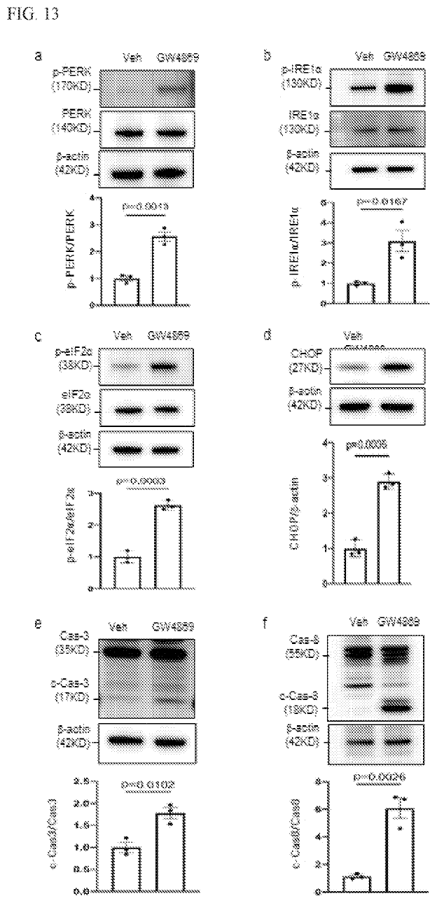

An exosome inhibitor mimics maternal diabetes by inducing NTDs. To determine whether exosomes are essential for neurulation, the exosome inhibitor GW4869 [41] was injected into dams from E7.0 to E8.5. Maternally injected GW4869 reached the embryo as indicated by GW4869 detection in the embryo (FIG. 7 L). GW4869 inhibited exosome production in the developing embryos (FIGS. 7 I and J) and mimicked the teratogenic effect of maternal diabetes on NTD induction (FIGS. 7 K and M, Table 6). Exosome inhibition by GW4869 triggered ER stress in neurulation stage embryos and neuroepithelial cell apoptosis (FIG. 13 A to F), key cellular events leading to failed neurulation.

TABLE 6

| | Glucose | | | NTD |
|---|---|---|---|---|
| Experimental | level | | | Embryos |
| group | (mg/dl) | Genotype | Embryos | (NTD rate) |
| ND-WT male x | 128.6 ± 31.6 | GW4869 | 96 | 7 (7.29%) * |
| WT female | | | | |
| (15 litters) | | | | |
| ND-WT male x | 121.38 ± 8.78 | vehicle | 62 | 0 (0.0%) |
| WT female | | | | |
| (8 litters) | | | | |

NTD, neural tube defect;
ND: nondiabetic;
WT: Wild-Type.
* Indicates significant difference (P < 0.05) when compared to other groups analyzed by the Fisher's Exact test.

Thus, a previously unappreciated crosstalk between the Flk-1⁺ progenitors and neuroepithelial cells was revealed, and survivin-contained exosomes produced by Flk-1⁺ progenitors mediate this crosstalk (FIG. 7N). The functional exosomal cargo survivin is transcriptionally regulated by FGF2, which itself is controlled by DNA methylation (FIG. 7N). Deviation of this crosstalk via fgf2 promoter hypermethylation leads to NTDs in diabetic pregnancy (FIG. 7N).

As shown herein, a mechanism underlying maternal diabetes-induced NTDs is revealed. During embryogenesis, proper communication among the three germ layers through exosomes is required for vasculogenesis and neurulation. The growth factor FGF2, produced by the endoderm, exerts its effect on Flk-1⁺ progenitors in the mesoderm, leading to the release of Survivin-enriched exosomes. These exosomes act on the neuroectoderm to prevent cellular stress and neuroepithelial cell apoptosis. Blocking this pathway under maternal diabetic conditions results in vasculopathy, which, in turn, contributes to NTD formation.

It has thus been established that crosstalk occurs among components of the endoderm, mesoderm and neuroectoderm via the growth factor FGF2 and the vascular progenitor specific factor Survivin-containing exosomes. This crosstalk is essential for successful neurulation. Maternal diabetes disrupts this crosstalk by suppressing FGF2 expression in the endoderm and FGF2 reduction leads to the inhibition of Survivin expression in the Flk-1⁺ vascular progenitors of the mesoderm. In diabetic pregnancy, the exosomes derived from Flk-1⁺ progenitors, which contain significantly less Survivin, cannot propel neural tube closure, leading to NTD formation. These findings reveal an important role for the Survivin-containing exosomes produced by Flk-1⁺ progenitors in neurulation, and indicate that the endoderm factor FGF2 sustains Survivin expression, a process that is compromised by maternal diabetes.

Exosomes enable cell-to-cell communication in spatially distinct tissues or organs. Indeed, exosomes derived from Flk-1⁺ progenitors, which exclusively reside in the yolk sac in early embryonic development, cross the intra-ammonic space to target cells in the neuroepithelium, which is the innermost layer of the embryo. This may explain why the Flk-1⁺ progenitor-specific factor Survivin plays a critical role in neurulation. It has been shown that deletion of the Survivin gene specifically in endothelial progenitors results in NTD formation [20]. Survivin is effectively packaged into Flk-1⁺ progenitor exosomes, and these Survivin-enriched exosomes are internalized by neuroepithelial cells. Thus, Survivin-enriched exosomes facilitate communication between Flk-1⁺ progenitors and the neuroepithelium and ensure the completion of proper neural tube closure.

The exosomal intercellular communication established herein clearly shows specificity. Neural stem cells cannot effectively take up exosomes derived from themselves. On the other hand, Flk-1⁺ progenitor exosomes are effectively internalized by neural stem cells or neuroepithelial cells. When Flk-1⁺ progenitor exosomes are delivered to the inside of the amniotic sac, these exosomes specifically enter neuroepithelial cells and significantly reduce NTD formation in diabetic pregnancy. Thus, Flk-1⁺ exosomes specifically target neuroepithelial cells of the developing embryo and can be used for the delivery of genes and other functional cargos into these cells.

While the invention has been described with reference to certain particular embodiments thereof, those skilled in the art will appreciate that various modifications may be made without departing from the spirit and scope of the invention. The scope of the appended claims is not to be limited to the specific embodiments described.

### REFERENCES

All patents and publications mentioned in this specification are indicative of the level of skill of those skilled in the art to which the invention pertains. Each cited patent and publication is incorporated herein by reference in its entirety. All of the following references have been cited in this application:
1 Mathews, T. J. & Driscoll, A. K. Trends in Infant Mortality in the United States, 2005-2014. NCHS Data Brief, 1-8 (2017).
2 Wallingford, J. B., Niswander, L. A., Shaw, G. M. & Finnell, R. H. The continuing challenge of understanding, preventing, and treating neural tube defects. Science 339, 1222002, doi:10.1126/science.1222002 (2013).
3 Correa, A. et al. Diabetes mellitus and birth defects. American J. Obstetrics and Gynecology 199, 237 e231-239 (2008).
4 Yang, P. et al. Maternal hyperglycemia activates an ASK1-FoxO3a-caspase 8 pathway that leads to embryonic neural tube defects. Science Signaling 6, ra74, doi:10.1126/scisignal.2004020 (2013).
5 Greene, M. F., Hare, J. W., Cloherty, J. P., Benacerraf, B. R. & Soeldner, J. S. First-trimester hemoglobin A1 and risk for major malformation and spontaneous abortion in diabetic pregnancy. Teratology 39, 225-231, doi:10.1002/tera.1420390303 (1989).
6 Lucas, M. J., Leveno, K. J., Williams, M. L., Raskin, P. & Whalley, P. J. Early pregnancy glycosylated hemoglobin, severity of diabetes, and fetal malformations. American J. Obstetrics and Gynecology 161, 426-431, doi:10.1016/0002-9378(89)90536-x (1989).
7 Suhonen, L., Hiilesmaa, V. & Teramo, K. Glycaemic control during early pregnancy and fetal malformations in women with type I diabetes mellitus. Diabetologia 43, 79-82, doi:10.1007/s001250050010 (2000).
8 Wang, F., Fisher, S. A., Zhong, J., Wu, Y. & Yang, P. Superoxide Dismutase 1 In Vivo Ameliorates Maternal Diabetes Mellitus-Induced Apoptosis and Heart Defects Through Restoration of Impaired Wnt Signaling. Circulation. Cardiovascular Genetics 8, 665-676, doi:10.1161/CIRCGENETICS.115.001138 (2015).
9 Yang, P. & Reece, E. A. Role of HIF-1alpha in maternal hyperglycemia-induced embryonic vasculopathy. American J. Obstetrics and Gynecology 204, 332 e331-337, doi:10.1016/j.ajog.2011.01.012 (2011).
10 Shalaby, F. et al. Failure of blood-island formation and vasculogenesis in Flk-1-deficient mice. Nature 376, 62-66, doi:10.1038/376062a0 (1995).
11 Yang, P. et al. Tip60- and sirtuin 2-regulated MARCKS acetylation and phosphorylation are required for diabetic embryopathy. Nat Commun 10, 282, doi:10.1038/s41467-018-08268-6 (2019).
12 Fine, E. L., Horal, M., Chang, T. I., Fortin, G. & Loeken, M. R. Evidence that elevated glucose causes altered gene expression, apoptosis, and neural tube defects in a mouse model of diabetic pregnancy. Diabetes 48, 2454-2462, doi:10.2337/diabetes.48.12.2454 (1999).
13 Salbaum, J. M. & Kappen, C. Neural tube defect genes and maternal diabetes during pregnancy. Birth Defects Res A Clin Mol Teratol 88, 601-611, doi:10.1002/bdra.20680 (2010).
14 Berdahl, D. M., Blaine, J., Van Voorhis, B. & Dokras, A. Detection of enlarged yolk sac on early ultrasound is associated with adverse pregnancy outcomes. Fertility and Sterility 94, 1535-1537, doi:10.1016/j.fertnstert.2009.12.064 (2010).
15 Yang, P., Zhao, Z. & Reece, E. A. Blockade of c-Jun N-terminal kinase activation abrogates hyperglycemia-induced yolk sac vasculopathy in vitro. American J. Obstetrics and Gynecology 198, 321 e321-327, doi:10.1016/j.ajog.2007.09.010 (2008).
16 McGough, I. J. & Vincent, J. P. Exosomes in developmental signalling. Development 143, 2482-2493, doi:10.1242/dev.126516 (2016).
17 Saha, P. et al. Circulating exosomes derived from transplanted progenitor cells aid the functional recovery of ischemic myocardium. Sci Transl Med 11, doi:10.1126/scitranslmed.aau1168 (2019).
18 Wheatley, S. P. & Altieri, D. C. Survivin at a glance. J Cell Sci 132, doi:10.1242/jcs.223826 (2019).
19 Khan, S. et al. Survivin is released from cancer cells via exosomes. Apoptosis: an international journal on programmed cell death 16, 1-12, doi:10.1007/s10495-010-0534-4 (2011).
20 Zwerts, F. et al. Lack of endothelial cell survivin causes embryonic defects in angiogenesis, cardiogenesis, and neural tube closure. Blood 109, 4742-4752, doi:10.1182/blood-2006-06-028068 (2007).
21 Wang, F. et al. Ask1 gene deletion blocks maternal diabetes-induced endoplasmic reticulum stress in the developing embryo by disrupting the unfolded protein response signalosome. Diabetes 64, 973-988, doi:10.2337/db14-0409 (2015).
22 Wang, F. et al. Protein kinase C-alpha suppresses autophagy and induces neural tube defects via miR-129-2 in diabetic pregnancy. Nat Commun 8, 15182, doi:10.1038/ncomms15182 (2017).
23 Pavlinkova, G., Salbaum, J. M. & Kappen, C. Maternal diabetes alters transcriptional programs in the developing embryo. BMC Genomics 10, 274, doi:10.1186/1471-2164-10-274 (2009).
24 Wentzel, P, Gareskog, M. & Eriksson, U. J. Decreased cardiac glutathione peroxidase levels and enhanced mandibular apoptosis in malformed embryos of diabetic rats. Diabetes 57, 3344-3352, doi:10.2337/db08-0830 (2008).
25 Kodet, O. et al. Melanoma cells influence the differentiation pattern of human epidermal keratinocytes. Mol Cancer 14, 1, doi:10.1186/1476-4598-14-1 (2015).
26 Yang, P., Zhao, Z. & Reece, E. A. Activation of oxidative stress signaling that is implicated in apoptosis with a mouse model of diabetic embryopathy. American J. Obstetrics and Gynecology 198, 130 e131-137, doi:10.1016/j.ajog.2007.06.070 (2008).
27 Doetschman, T., Shull, M., Kier, A. & Coffin, J. D. Embryonic stem cell model systems for vascular morphogenesis and cardiac disorders. Hypertension 22, 618-629, doi:10.1161/01.hyp.22.4.618 (1993).
28 Kessler, D. S. & Melton, D. A. Vertebrate embryonic induction: mesodermal and neural patterning. Science 266, 596-604, doi:10.1126/science.7939714 (1994).
29 Zhong, J., Xu, C., Reece, E. A. & Yang, P. The green tea polyphenol EGCG alleviates maternal diabetes-induced neural tube defects by inhibiting DNA hypermethylation. American J. Obstetrics and Gynecology 215, 368 e361-368 e310, doi:10.1016/j.ajog.2016.03.009 (2016).
30 Wei, D. & Loeken, M. R. Increased DNA methyltransferase 3b (Dnmt3b)-mediated CpG island methylation stimulated by oxidative stress inhibits expression of a gene required for neural tube and neural crest development in diabetic pregnancy. Diabetes 63, 3512-3522, doi:10.2337/db14-0231 (2014).
31 Yu, J., Wu, Y. & Yang, P. High glucose-induced oxidative stress represses sirtuin deacetylase expression and increases histone acetylation leading to neural tube defects. J Neurochem 137, 371-383, doi:10.1111jnc.13587 (2016).
32 Yang, P. & Li, H. Epigallocatechin-3-gallate ameliorates hyperglycemia-induced embryonic vasculopathy and malformation by inhibition of Foxo3a activation. American J. Obstetrics and Gynecology 203, 75 e71-76, doi:10.1016/j.ajog.2010.02.008 (2010).
33 Poole, T. J., Finkelstein, E. B. & Cox, C. M. The role of FGF and VEGF in angioblast induction and migration during vascular development. Developmental dynamics: an official publication of the American Association of Anatomists 220, 1-17, doi: 10.1002/1097-0177(2000)9999:9999<::AID-DVDY1087>3.0.CO; 2-2 (2001).
34 Flamme, I. & Risau, W. Induction of vasculogenesis and hematopoiesis in vitro. Development 116, 435-439 (1992).
35 Brueckner, B. et al. Epigenetic reactivation of tumor suppressor genes by a novel small-molecule inhibitor of human DNA methyltransferases. Cancer Res 65, 6305-6311, doi:10.1158/0008-5472.CAN-04-2957 (2005).
36 Li, X., Xu, C. & Yang, P. c-Jun NH2-terminal kinase 1/2 and endoplasmic reticulum stress as interdependent and reciprocal causation in diabetic embryopathy. Diabetes 62, 599-608, doi:10.2337/db12-0026 (2013).
37 Wang, F., Reece, E. A. & Yang, P. Superoxide dismutase 1 overexpression in mice abolishes maternal diabetes-induced endoplasmic reticulum stress in diabetic embryopathy. American J. Obstetrics and Gynecology 209, 345 e341-347, doi:10.1016/j.ajog.2013.06.037 (2013).
38 Ambrosini, G., Adida, C. & Altieri, D. C. A novel anti-apoptosis gene, survivin, expressed in cancer and lymphoma. Nature Medicine 3, 917-921 (1997).
39 Dohi, T., Beltrami, E., Wall, N. R., Plescia, J. & Altieri, D. C. Mitochondrial survivin inhibits apoptosis and promotes tumorigenesis. The Journal of Clinical Investigation 114, 1117-1127, doi:10.1172/JCI22222 (2004).
40 Tkach, M. & Thery, C. Communication by Extracellular Vesicles: Where We Are and Where We Need to Go. Cell 164, 1226-1232, doi:10.1016/j.cell.2016.01.043 (2016).
41 Seo, N. et al. Activated CD8(+) T cell extracellular vesicles prevent tumour progression by targeting of lesional mesenchymal cells. Nat Commun 9, 435, doi:10.1038/s41467-018-02865-1 (2018).
42 Srivastava, A. et al. Exploitation of Exosomes as Nanocarriers for Gene-, Chemo-, and Immune-Therapy of Cancer. J Biomed Nanotechnol 12, 1159-1173, doi:10.1166/jbn.2016.2205 (2016).
43 Rana, S., Yue, S., Stadel, D. & Zoller, M. Toward tailored exosomes: the exosomal tetraspanin web contributes to target cell selection. Int J Biochem Cell Biol 44, 1574-1584, doi:10.1016/j.biocel.2012.06.018 (2012).
44 Ronicke, V., Risau, W. & Breier, G. Characterization of the endothelium-specific murine vascular endothelial growth factor receptor-2 (Flk-1) promoter. Circulation Research 79, 277-285, doi:10.1161/01.res.79.2.277 (1996).
45 Xu, C., Li, X., Wang, F., Weng, H. & Yang, P. Trehalose prevents neural tube defects by correcting maternal diabetes-suppressed autophagy and neurogenesis. American J. of Physiology. Endocrinology and Metabolism 305, E667-678, doi:10.1152/ajpendo.00185.2013 (2013).
46 Li, X., Weng, H., Xu, C., Reece, E. A. & Yang, P. Oxidative stress-induced JNK1/2 activation triggers proapoptotic signaling and apoptosis that leads to diabetic embryopathy. Diabetes 61, 2084-2092, doi:10.2337/db11-1624 (2012).

1. A method of preventing neural tube defects (NTDs), reducing diabetes-induced neuroepithelial cell apoptosis, and/or abrogating maternal diabetes-induced cellular stress in an embryo of a pregnant diabetic female subject, said method comprising administering to a pregnant diabetic female subject and/or an embryo of a pregnant diabetic female subject a therapeutically-effective amount of exosomes containing Survivin polypeptide.

2-3. (canceled)

4. The method of claim 1, wherein the NTD is failure of neural tube closure.

5. The method of claim 1, wherein the embryo has an age of from about E7 to E9.

6. The method of claim 1, wherein the exosomes are administered to the amniotic cavity of the embryo.

7. The method of claim 6, wherein the administration is via a microinjection.

8. The method of claim 1, wherein the exosomes are derived from Flk-1⁺ mesoderm progenitor cells.

9. The method of claim 1, wherein the therapeutically-effective amount of exosomes is a population of between 1×10⁶ and 1×10¹² exosomes.

10. The method of claim 1, wherein the female subject has gestational diabetes.

11. The method of claim 1, wherein the female subject is a female mammal.

12. A method of preventing NTDs, reducing diabetes-induced neuroepithelial cell apoptosis, and/or abrogating maternal diabetes-induced cellular stress in an embryo of a pregnant diabetic female subject, said method comprising administering to a pregnant diabetic female subject and/or an embryo of a pregnant diabetic female subject a therapeutically-effective amount of fibroblast growth factor 2 (FGF2).

13-14. (canceled)

15. The method of claim 12, wherein the NTD is failure of neural tube closure.

16. The method of claim 12, wherein the embryo has an age of from about E7 to E9.

17. The method of claim 12, wherein the FGF2 is administered to the amniotic cavity of the embryo or the neuroepithelium of the embryo.

18. The method of claim 17, wherein the administration is via a microinjection.

19. The method of claim 12, wherein the therapeutically-effective amount of FGF2 is between 1 ng and 500 ng of FGF2.

20. The method of claim 12, wherein the female subject has gestational diabetes.

21. The method of claim 12, wherein the female subject is a female mammal.

22. A method for delivering a biological molecule to neuroepithelial cells of an embryo of a pregnant female subject, said method comprising administering to a pregnant female subject and/or an embryo of a pregnant female subject a therapeutically-effective amount of exosomes containing a biological molecule.

23. (canceled)

24. The method of claim 22, wherein the embryo has an age of from about E7 to E9.

25. The method of claim 24, wherein the administration is via a microinjection.

26. The method of claim 22, wherein the female subject has gestational diabetes.

27. The method of claim 22, wherein the female subject is a female mammal.
